(19) United States
(12) Patent Application Publication
(10) Pub. No.: US 2025/0177318 A1
(43) Pub. Date: Jun. 5, 2025

(54) CMT-3 FORMULATIONS AND METHODS OF USING THE SAME

(71) Applicant: The Research Foundation for the State University of New York, Albany, NY (US)

(72) Inventors: Juntao LUO, Jamesville, NY (US); Robert COONEY, Syracuse, NY (US); Qinghe MENG, East Syracuse, NY (US); Xiaojing WANG, East Syracuse, NY (US); Dandan GUO, Syracuse, NY (US); Gary NIEMAN, Syracuse, NY (US); Lorne GOLUB, Smithtown, NY (US); Francis JOHNSON, Setauket, NY (US); Hsi-Ming LEE, Setauket, NY (US)

(21) Appl. No.: 18/726,929

(22) PCT Filed: Jan. 3, 2023
(86) PCT No.: PCT/US2023/010041

§ 371 Date: Jul. 5, 2024

Related U.S. Application Data
(60) Provisional application No. 63/296,420, filed on Jan. 4, 2022.
(60) Provisional application No. 63/296,389, filed on Jan. 4, 2022.

(51) Int. Cl. A61K 9/51; A61K 9/00; A61K 9/12; A61K 31/65; A61P 11/00

(52) U.S. Cl. CPC A61K 9/5146; A61K 9/0019; A61K 9/0078; A61K 9/12; A61K 31/65; A61P 11/00

(57) ABSTRACT

Provided are functional segregated telodendrimers having, for example, two or three functional segments. The telodendrimers can aggregate to form nanocarriers. The telodendrimers can have one or more tetracycline drugs (such as CMT-3) physically bound thereto that release under physiological conditions. Such nanocarriers with loaded CMT-3 disperse stably in aqueous solution and may be used in treating inflammatory disease through local or systemic administration. Also provided are methods of treating acute respiratory distress syndrome (ARDS) and/or CARDS in a subject in need thereof, including: administering a therapeutically effective amount of CMT-3 and one or more nonantimicrobial host-modulators directly to the lungs of the patient. In embodiments, CMT-3 is administered to the lungs in an aerosolized formulation. In embodiments, subsequent to CMT-3 administration, one or more non-antimicrobial host-modulators directly to the lungs of the patient.

### CROSS-REFERENCE TO RELATED APPLICATIONS

This application claims the benefit of the filing date of U.S. Provisional Applications Nos. 63/296,389 and 63/296,420, both of which were filed Jan. 4, 2022. The content of these earlier filed applications are hereby incorporated by reference herein in their entirety.

### STATEMENT REGARDING FEDERALLY SPONSORED RESEARCH OR DEVELOPMENT

This invention was made with government support under grant no. GM130941 awarded by the National Institutes of Health. The government has certain rights in the invention.

### FIELD OF THE INVENTION

This disclosure generally relates to telodendrimers, and methods of making and using telodendrimers. More particularly, the disclosure relates to functional segregated telodendrimers suitable for use in CMT-3 nanocarrier formulations. In embodiments, the present disclosure relates to CMT-3 nanocarrier formulation for systemic and local administration for inflammation control and/or treatment of inflammatory disease of the lungs, other organs, and the like. In embodiments, the present disclosure relates to an aerosolized drug delivery system suitable of administering CMT-3 nanocarrier formulations of the present disclosure to a subject in need thereof.

### BACKGROUND

Bacterial or viral pneumonia, and acute lung injury (ALI) lead to acute respiratory distress syndrome (ARDS) and systemic inflammation. CMT-3 has pleiotropic anti-inflammatory effects including matrix metalloproteinase (MMP) inhibition, attenuating neutrophil (PMN) activation, and elastase release. Unfortunately, CMTs are only available for oral administration and their hydrophobicity, poor solubility, and limited absorption through the intestinal tract limit their efficacy in ALW/ARDS. Accordingly, administration or delivery of CMT-3 is problematic in targeting lung cells and tissues in need thereof and deficient in providing any significant clinical benefit.

Prior art of interest includes U.S. Pat. No. 5,977,091 entitled Method of Preventing Acute Lung Disease; U.S. Patent Publication No. US20040092491 entitled Method of Treating Sepsis-induced ARDS; U.S. Pat. No. 5,773,430 entitled Serine Proteinase Inhibitory Activity by Hydrophobic Tetracycline; U.S. Patent Publication No. 2008/0233151 entitled Use of Non-antibacterial Tetracycline Analogs and Formulations thereof for the Treatment of Bacterial Exotoxins; and U.S. Patent Application No. 2015/0056139 entitled Telodendrimers and Nanocarriers and Methods of Using Same to Luo et al. (all of which are herein entirely incorporated entirely by reference). However, the process sequences and drug delivery embodiments of these references are different than the present disclosure and fail to provide CMT-3 and/or analogues thereof to a subject in need thereof in a safe and efficacious manner.

There is a continuous need for compositions, methods and drug delivery systems for targeted drug delivery to organs, tissues and cells in need thereof that overcome these limitations while being safe and efficacious. Further, there is a desire for robust, stable, pharmaceutically acceptable formulations for drug delivery, such as CMT-3 delivery via non-oral administration.

### SUMMARY

In embodiments, the present disclosure provides compositions, methods, and drug delivery systems for targeted drug delivery resulting in significant clinical benefits in disease treatment, especially for lung disease or trauma such as intratracheal lipopolysaccharide (LPS) induced acute lung injury (ALI). Encapsulation of anti-inflammatory drugs such as CMT-3 inside or as a nanoparticle (nCMT-3) decreases side toxicity and improves the life quality of a subject in need thereof. In addition, passive or active targeting effect of a nanocarrier of the present disclosure delivers a significantly high dose of drugs, such as CMTs, to subjects in need thereof and yields improved treatment for trauma or disease. In embodiments, an aerosolized drug delivery system including one or more nanocarriers of the present disclosure yields improved organ or tissue treatment for trauma or disease, including, but not limited to lung trauma or disease.

Embodiments of the present disclosure provide pharmaceutically acceptable stable formulations with excellent drug loading capacity, reproducibility and biocompatibility. In embodiments, intratracheal nCMT-3 administration in accordance with the present disclosure attenuates LPS-induced ALI. In embodiments, intratracheal nCMT-3 administration in accordance with the present disclosure attenuates LPS-induced ALI by attenuating MMP activation, sTREM-1 release and NLRP3 inflammasome activation.

In some embodiments, the present disclosure provides a nanocarrier composition, including: one or more telodendrimers suitable for binding to one or more tetracyclines; and one or more tetracyclines have substantially no antibacterial activity. In embodiments, the tetracycline is 6-demethyl-6-deoxy-4-de(dimethylamino)tetracycline (CMT-3), or a derivative or analogue thereof.

In some embodiments, the present disclosure provides a method for preventing, alleviating, or treating acute lung injury or disease in a mammal, the method including: administering a nanocarrier composition to a mammal in need thereof in a therapeutically effective amount, wherein the nanocarrier composition includes one or more telodendrimers suitable for binding to one or more tetracyclines, wherein the one or more tetracyclines have substantially no antibacterial activity. In embodiments, the tetracycline is 6-demethyl-6-deoxy-4-de(dimethylamino)tetracycline (CMT-3), or a derivative or analogue thereof.

In some embodiments, the present disclosure provides a method for treating inflammation or inflammatory disease in a mammal, the method including: administering a nanocarrier composition to a mammal in need thereof in a therapeutically effective amount, wherein the nanocarrier composition includes one or more telodendrimers suitable for binding to one or more tetracyclines, wherein the one or more tetracyclines have substantially no antibacterial activity. In embodiments, the tetracycline is 6-demethyl-6-deoxy-4-de(dimethylamino)tetracycline (CMT-3), or a derivative or analogue thereof.

In some embodiments, the present disclosure includes a nanocarrier composition, including: one or more telodendrimers suitable for binding to a tetracycline, wherein the tetracycline is 6-demethyl-6-deoxy-4-de(dimethylamino)tetracycline (CMT-3), or a derivative or analogue thereof, and wherein the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof.

In embodiments, the present disclosure includes an aerosolized drug delivery system, including a plurality of nanocarrier compositions of the present disclosure. In embodiments, the plurality of nanocarrier compositions include a drug such as CMT-3 in a therapeutically effective amount.

In embodiments, the present disclosure includes an injectable drug delivery system, including a plurality of nanocarrier compositions of the present disclosure. In embodiments, the plurality of nanocarrier compositions include a drug such as CMT-3 in a therapeutically effective amount.

In embodiments, the present disclosure includes a method of treating inflammation or inflammatory disease including: administering a nanocarrier composition to a mammal in need thereof in a therapeutically effective amount, wherein the nanocarrier composition includes one or more telodendrimers suitable for binding to one or more tetracyclines, wherein the one or more tetracyclines have substantially no antibacterial activity. In embodiments the nanocarrier composition targets, dental, lung, liver, kidney tissues, or combinations thereof. In embodiments, the tetracycline is 6-demethyl-6-deoxy-4-de(dimethylamino)tetracycline (CMT-3), or an analogue or derivative thereof. In embodiments, the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof.

In embodiments, the present disclosure includes a pharmaceutically acceptable composition for pulmonary inhalation, including: one or more nanocarriers including one or more telodendrimers suitable for binding to one or more tetracyclines, wherein the one or more tetracyclines have substantially no antibacterial activity. In embodiments, the composition is suitable for aerosolized delivery to a subject in need thereof. In embodiments, the composition is a dry powder.

In embodiments, the present disclosure includes a pharmaceutically acceptable composition for injectable administration, including: one or more nanocarriers comprising one or more telodendrimers suitable for binding to one or more tetracyclines, wherein the one or more tetracyclines have substantially no antibacterial activity. In embodiments, the composition is a liquid, and includes a therapeutically effective amount of tetracycline. In embodiments, the tetracycline is characterized as an anti-inflammatory.

In embodiments, the present disclosure includes an aerosolized drug delivery system, including: a therapeutically effective amount of CMT-3.

In embodiments, the present disclosure includes an injectable drug delivery system, including: a therapeutically effective amount of CMT-3.

In embodiments, the present disclosure includes a pharmaceutically acceptable composition for pulmonary inhalation, including: one or more tetracyclines, wherein the one or more tetracyclines have substantially no antibacterial activity. In embodiments, the composition is suitable for aerosolized delivery to a subject in need thereof. In embodiments, the composition is a dry powder. In embodiments, the one or more tetracycline is CMT-3.

In embodiments, the present disclosure includes a pharmaceutically acceptable composition for injectable administration, including: one or more tetracyclines having substantially no antibacterial activity. In embodiments, the composition is a liquid, and includes a therapeutically effective amount of tetracycline. In embodiments, the tetracycline is characterized as an anti-inflammatory. In embodiments, the tetracycline is CMT-3.

The illustrative aspects of the present disclosure are designed to solve the problems herein described and/or other problems not discussed.

### BRIEF DESCRIPTION OF THE DRAWINGS

FIGS. 1A, 1B, and 1C are telodendrimer embodiments of the present disclosure such as PEG^{nk}CA₄VE₄, wherein n=5, PEG^{nk}CA₄URA₄, wherein n=5, and PEG^{nk}CA₄-L-VE₄, respectively.

FIG. 2 is an example of a synthetic route embodiment of the present disclosure for the formation of PEG^{nk}CA₄VE₄, wherein n=5.

FIG. 3 is an example of a synthetic route embodiment of the present disclosure for the formation of PEG^{nk}CA₄URA₄, wherein n=5.

FIG. 4 is an example of a synthetic route embodiment of the present disclosure for the formation of PEG^{nk}CA₄-L-VE₄.

FIGS. 5A-5D depict in vitro characterization of CMT-3-PEG^{5k}CA₄VE₄ nanoformulation.

FIGS. 6A-6B depict in vivo pharmacokinetics and biodistribution of CMT-3-PEG^{5k}CA₄VE₄ nanoformulation after (FIG. 6A) intravenous injection (IV) or (FIG. 6B) intratracheal injection (IT): CMT-3 concentration in plasma and major organs (liver, lungs, kidney, spleen and heart) was analyzed by HPLC after solid-phase extraction.

FIGS. 7A-7F depict matrix metallopeptidase 2 & 9 (MMP-2 & 9) levels lung tissue and the levels of neutrophil elastase (NE) in blood and BALF.

FIGS. 8A-8D depict hematological analysis in accordance with the present disclosure.

FIGS. 9A-9E depict cytokine levels in bronchoalveolar lavage fluid (BALF) and plasma.

FIGS. 10A-10D depict a cytological analysis and total protein concentration in bronchoalveolar lavage fluid (BALF).

FIGS. 11A and 11B depict a histological assessment of lung injury.

FIGS. 12A and 12B depict the levels of soluble triggering receptor expressed on myeloid cells 1 (sTREM-1) in blood and BALF.

FIGS. 13A and 13B depict the levels of caspase-1 and NPRL3 inflammasome in lung tissue.

FIGS. 14A and 14B depict the effect of telodendrimer (PEG^{5K}CA₄VE₄) (Nano) on neutrophil in LPS-induced lung injury in mice.

FIG. 15 depicts a drug release profile of CMT-3-PEG^{5k}CA₄VE₄, CMT-3-PEG^{20k}CA₄VE₄, CMT-3-PEG^{5k}CA₄-L-VE₄ in PBS at 37° C.

FIGS. 16A-16E depict in vitro characterization of a CMT-3-PEG^{5k}CA₄VE₄ nanoformulation.

FIGS. 17A-17D depict PEG^{nk}CA₄VE₄, PEG^{nk}CA₄URA₄, PEG^{nk}CA₄-L-VE₄, respectively, and moieties thereof (FIG. 17D).

FIG. 18 depicts a nanocarrier of the present disclosure including PEG^{nk}CA₄VE₄, PEG^{nk}CA₄URA₄, PEG^{nk}CA₄-L-VE₄ and CMT-3.

FIGS. 19A-19D depict another hematological analysis in accordance with the present disclosure.

FIGS. 20A and 20B depict the effect of telodendrimer (PEG^{5K}CA₄VE₄) (Nano) on NLR (%) and IL-6 in LPS-induced lung injury in mice.

FIGS. 21A-21F depict the effect of telodendrimer (PEG^{5K}CA₄VE₄) (Nano) on neutrophil in LPS-induced lung injury in mice.

FIGS. 22A-22G depict aerosolized nCMT-3 delivery to lungs.

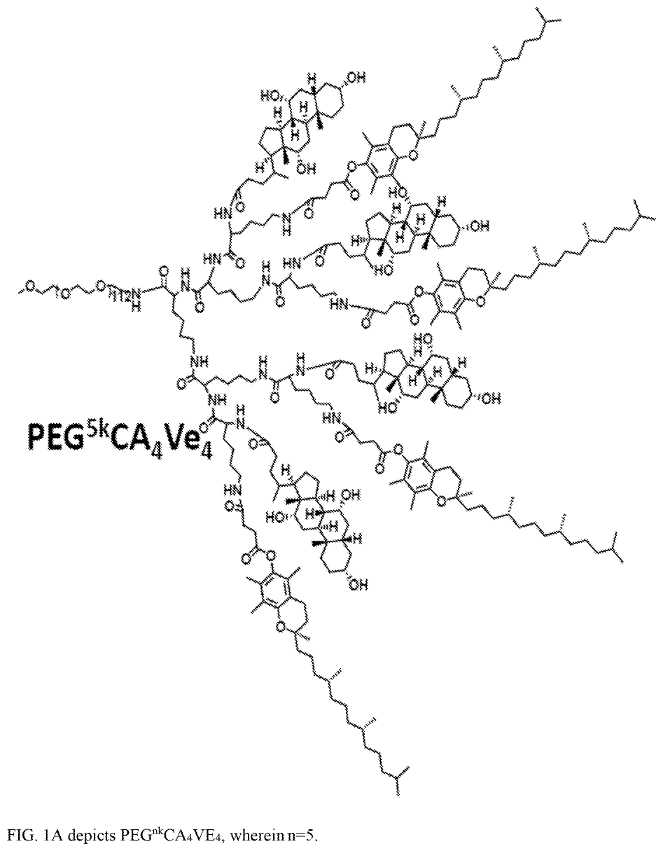

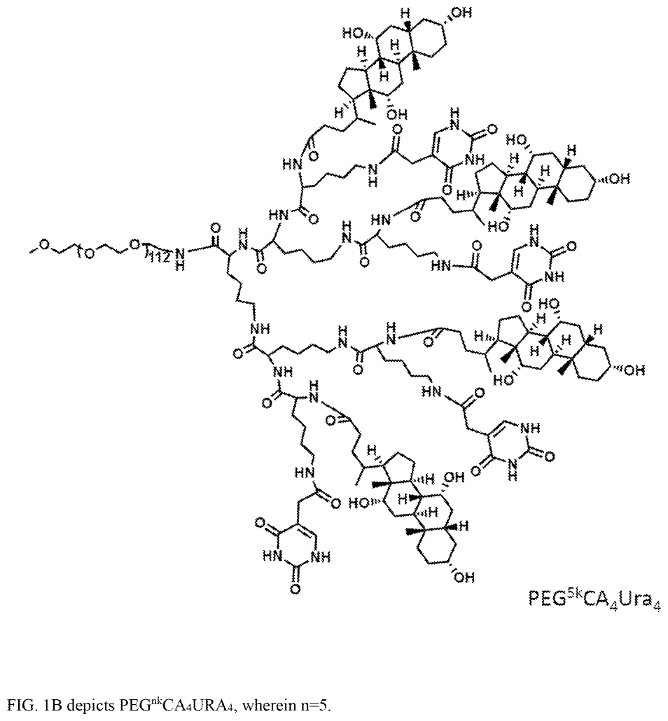

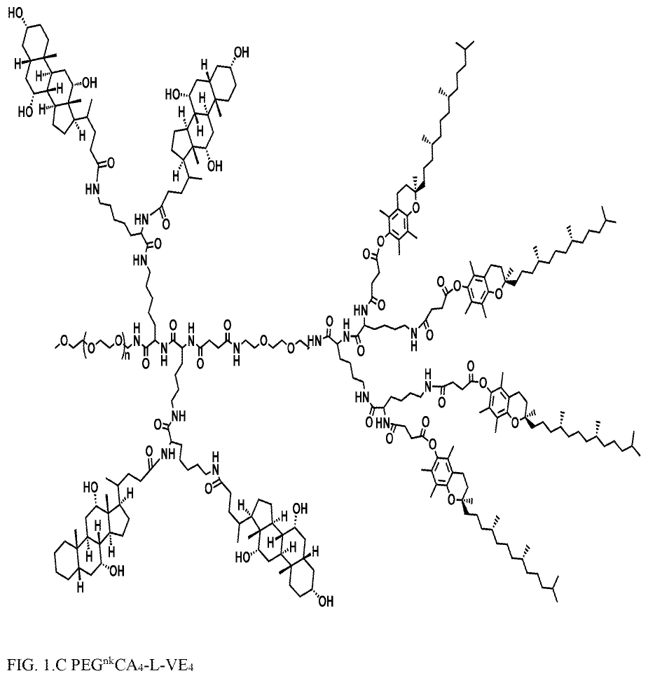

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION OF THE DISCLOSURE

The present disclosure relates to methods and compositions for treating inflammatory disease, including inflammatory lung disease. For example, in embodiments, the present disclosure provides a nanocarrier composition, including: one or more telodendrimers suitable for binding to one or more tetracyclines; and one or more tetracyclines have substantially no antibacterial activity. In embodiments, the tetracycline is 6-demethyl-6-deoxy-4-de(dimethylamino)tetracycline (CMT-3), or a derivative thereof. In embodiments, the tetracycline is man-made and/or non-naturally occurring. In embodiments, a tetracycline or drug, or active agent is able to physically bind to the one or more telodendrimers of the present disclosure and release under physiological conditions such as within a subject in need thereof, and, in embodiments, at a tissue or organ in need thereof.

In embodiments, the present disclosure provides targeted drug delivery methods and compositions resulting in significant clinical benefits for disease treatment, especially for lung disease or trauma such as LPS-induced ALI. Encapsulation of anti-inflammatory drugs inside a nanoparticle decreases side toxicity and improves the life quality of a subject in need thereof. In embodiments, a drug, such as CMT-3 physically binds to one or telodendrimers of the present disclosure, and release therefrom in a physiological acceptable environment such as within a subject in need thereof. In addition, passive or active targeting effect of the nanocarrier is able to deliver significantly high dose of drugs to cells and tissues and yields improved tissue or organ treatment such as lung treatment. Advantages of embodiments of the present disclosure include: pharmaceutically acceptable stable formulations with excellent drug loading capacity, reproducibility, and biocompatibility. In embodiments, intratracheal nCMT-3 administration attenuates LPS-induced ALI by attenuating MMP activation, sTREM-1 release and NLRP3 inflammasome activation.

In some embodiments, the present disclosure provides a method of treating acute respiratory distress syndrome (ARDS) and/or CARDS in a subject in need thereof, including: administering a therapeutically effective amount of CMT-3 and one or more non-antimicrobial host-modulators directly to the lungs of the patient. In embodiments, the CMT-3 is characterized as a pleiotropic anti-inflammatory drug. In embodiments, the subject has COVID-19. In embodiments, CMT-3 is administered via an aerosolized drug delivery system, including a therapeutically effective amount of CMT-3. In embodiments, CMT-3 is administered via a pharmaceutically acceptable composition for pulmonary inhalation, including: one or more tetracyclines, wherein the one or more tetracyclines have substantially no antibacterial activity.

In some embodiments, the present disclosure includes an aerosolized drug delivery system, including a therapeutically effective amount of CMT-3. In some embodiments, the present disclosure includes an injectable drug delivery system, including: a therapeutically effective amount of CMT-3.

In embodiments, the present disclosure includes a pharmaceutically acceptable composition for pulmonary inhalation, including: one or more tetracyclines, wherein the one or more tetracyclines have substantially no antibacterial activity. In embodiments, the composition is suitable for aerosolized delivery to a subject in need thereof. In embodiments, the one or more tetracyclines is CMT-3.

In embodiments, the present disclosure includes a pharmaceutically acceptable composition for injectable administration, including: one or more tetracyclines having substantially no antibacterial activity. In embodiments, the composition is a liquid, and includes a therapeutically effective amount of tetracycline. In embodiments, the tetracycline is characterized as an anti-inflammatory. In embodiments, the tetracycline is CMT-3.

In some embodiments, the present disclosure includes a method of treating COVID-19 induced ARDS, including: administering CMT-3 to a subject or patient in need thereof for a first duration, and subsequent to the first duration, administering doxycline hyclate to the subject. In embodiments, CMT-3 is administered in a therapeutically effective amount. In embodiments, the doxycline hyclate is administered in a therapeutically effective amount. In embodiments, CMT-3 is administered in a therapeutically effective amount in an aerosolized formulation.

Definitions: As used herein, the singular forms “a”, “an”, and “the” include plural references unless the context clearly dictates otherwise. Thus, for example, references to “a composition” include the use of one or more compositions. “A step” of a method means at least one step, and it could be one, two, three, four, five or even more method steps.

The term “about”, as used herein, refers to +/−10% of the stated value or a chemical or obvious equivalent thereof.

As used herein the term “analog” includes compounds having structural or functional similarity to another compound. In embodiments, compounds having structural similarity to another (a parent compound) that mimic the biological or chemical activity of the parent compound are analogs. There are no minimum or maximum numbers of elemental or functional group substitutions required to qualify a compound as an analog provided the analog is capable of mimicking, in some relevant fashion, either identically, complementarily or competitively, with the biological or chemical properties of the parent compound. Analogs can be, and often are, derivatives of the parent compound (see “derivative”). Analogs of the compounds disclosed herein may have equal, lesser or greater activity than their parent compounds.

As used herein the term “derivative” refers to a compound made from (or derived from), either naturally or synthetically, a parent compound. A derivative may be an analog (see “analog” supra) and thus may possess similar chemical or biological activity. However, unlike an analog, a derivative does not necessarily have to mimic the biological or chemical activity of the parent compound. There are no minimum or maximum numbers of elemental or functional group substitutions required to qualify a compound as a derivative. In embodiments, derivatives of the compounds disclosed herein may have equal, less, greater or even distinct activities when compared to their parent compounds.

As used herein, the term “drug delivery system” refers to a system for delivering one or more active agents.

As used herein, the term “dry powder” refers to a fine particulate composition that is not suspended or dissolved in a propellant, liquid, or other carrier. It is not meant to necessarily imply a complete absence of all water molecules.

As used herein the term “pulmonary inhalation” is used to refer to administration of pharmaceutical preparations by inhalation so that they reach the lungs and in particular embodiments the alveolar regions of the lung. Typically inhalation is through the mouth, but in alternative embodiments in can entail inhalation through the nose. In embodiments, nCMT nanocarriers of the present disclosure are suitable for use in pulmonary inhalation.

As used herein, the term “effective amount” refers to that amount of a substance that is necessary or sufficient to bring about a desired biologic effect. An effective amount can but need not be limited to an amount administered in a single administration.

As used herein the term “polyethylene glycol” refers to a polyether compound commonly expressed as H—(O—CH₂—CH₂)ₙ—OH. In embodiments, polyethylene glycol may refer to polyethylene oxide (PEO) or polyoxyethylene (POE), depending on its molecular weight.

As used herein the term “pharmaceutically acceptable” substances refers to those substances which are within the scope of sound medical judgment suitable for use in contact with the tissues of subjects without undue toxicity, irritation, allergic response, and the like, and effective for their intended use. In embodiments, a nanocarrier composition of the present disclosure is characterized as pharmaceutically acceptable.

As used herein the term “pharmaceutical composition” refers to the combination of one or more substances such as e.g., one or more nanocarriers in accordance with the present disclosure and one or more excipients and one or more pharmaceutically acceptable vehicles with which the one or more nanocarriers in accordance with the present disclosure is administered to a subject.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater. In embodiments, nanocarrier material of the present disclosure is characterized as substantially purified. In embodiments, nCMT-3 is substantially purified.

As used herein, the term “telodendrimer” refers to a linear-dendritic copolymer, containing an optional hydrophilic segment (i.e., PEG moiety) and one or more chemical moieties covalently bonded to one or more end groups of the dendron. Suitable moieties include, but are not limited to, hydrophobic groups, hydrophilic groups, amphiphilic compounds, and drugs. Different moieties may be selectively installed at selected end groups using orthogonal protecting group strategies.

As used herein, the term “moiety” refers to a part (substructure) or functional group of a molecule that is part of the telodendrimer structure.

As used herein, the term “dendritic polymer” refers to branched polymers containing a focal point, a plurality of branched monomer units, and a plurality of end groups. The monomers are linked together to form arms (or “dendritic polymer”) extending from the focal point and terminating at the end groups. The focal point of the dendritic polymer can be attached to other segments of the compounds of the disclosure, and the end groups may be further functionalized with additional chemical moieties.

As used herein, the term “nanocarrier” refers to a micelle resulting from aggregation of telodendrimer conjugates of the present disclosure. The nanocarrier has a hydrophobic core and a hydrophilic exterior.

As used herein, the terms “monomer” and “monomer unit” refer to a diamino carboxylic acid, a dihydroxy carboxylic acid, or a hydroxylamino carboxylic acid. Examples of diamino carboxylic acid groups of the present disclosure include, but are not limited to, 2,3-diamino propanoic acid, 2,4-diaminobutanoic acid, 2,5-diaminopentanoic acid (ornithine), 2,6-diaminohexanoic acid (lysine), (2-aminoethyl)-cysteine, 3-amino-2-aminomethyl propanoic acid, 3-amino-2-aminomethyl-2-methyl propanoic acid, 4-amino-2-(2-aminoethyl) butyric acid and 5-amino-2-(3-aminopropyl)pentanoic acid. Examples of dihydroxy carboxylic acid groups of the present disclosure include, but are not limited to, glyceric acid, 2,4-dihydroxybutyric acid, glyceric acid, 2,4-dihydroxybutyric acid, 2,2-bis(hydroxymethyl)propionic acid, and 2,2-bis(hydroxymethyl)butyric acid. Examples of hydroxyl amino carboxylic acids include, but are not limited to, serine and homoserine. One of skill in the art will appreciate that other monomer units can be used in the present disclosure.

As used herein, the term “linker” or an “L” group refers to a chemical moiety that links (e.g., via covalent bonds) one segment of a dendritic conjugate to another segment of the dendritic conjugate. The types of bonds used to link the linker to the segments of the telodendrimers include, but are not limited to, amides, amines, esters, carbamates, ureas, thioethers, thiocarbamates, thiocarbonate, and thioureas. For example, in embodiments, a linker individually at each occurrence in the telodendrimer, can be a polyethylene glycol moiety, polyserine moiety, polyglycine moiety, poly(serine-glycine) moiety, aliphatic amino acid moieties, 6-amino hexanoic acid moiety, 5-amino pentanoic acid moiety, 4-amino butanoic acid moiety, and beta-alanine moiety. In embodiments, a linker can also be a cleavable linker. In certain embodiments, combinations of linkers can be used. For example, the linker can be an enzyme cleavable peptide moiety, disulfide bond moiety or an acid labile moiety. One of skill in the art will appreciate that other types of bonds can be used in the present disclosure.

As used herein, the term “oligomer” refers to fifteen or fewer monomers, as described above, covalently linked together. The monomers may be linked together in a linear or branched fashion. The oligomer may function as a focal point for a branched segment of a telodendrimer.

As used herein, the term “hydrophobic group” refers to a chemical moiety that is water-insoluble or repelled by water. Examples of hydrophobic groups include, but are not limited to, long-chain alkanes and fatty acids, fluorocarbons, silicones, certain steroids such as, for example, cholesterol, and certain polymers such as, for example, polystyrene and polyisoprene.

As used herein, the term “hydrophilic group” refers to a chemical moiety that is water-soluble or attracted to water. Examples of hydrophilic groups include, but are not limited to, alcohols, short-chain carboxylic acids, quaternary amines, sulfonates, phosphates, sugars, and certain polymers such as, for example, PEG.

As used herein, the term “amphiphilic compound” refers to a compound having both hydrophobic portions and hydrophilic portions. For example, the amphiphilic compounds of the present disclosure can have one hydrophilic face of the compound and one hydrophobic face of the compound.

As used herein, the terms “drug” or “therapeutic agent” refers to an agent capable of treating and/or ameliorating a condition or disease. A drug may be a hydrophobic drug, which is any drug that repels water. Hydrophobic drugs useful in the present disclosure include, but are not limited to, paclitaxel, doxorubicin, etoposide, irinotecan, SN-38, cyclosporin A, podophyllotoxin, Carmustine, Amphotericin (Amphotericin B), Ixabepilone, Patupilone (epothelone class), rapamycin, bortezomib, gambogic acid, oridonin, norcantharidin, triptolide, camptothecin, docetaxel, daunorubicin, VP 16, prednisone, methotrexate, dexamethasone, vincristine, vinblastine, temsirolimus, and platinum drugs (e.g., cisplatin, carboplatin, oxaplatin). The drugs of the present disclosure also include prodrug forms and drug-like compounds. In embodiments, the drug of the present disclosure includes CMT-3, or analogues or derivatives thereof. One of skill in the art will appreciate that other drugs can be used in the present disclosure.

As used herein, the terms “treat”, “treating” and “treatment” refer to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the subject; decreasing the frequency or duration of the symptom or condition; or, in some situations, preventing the onset of the symptom or condition. The treatment or amelioration of symptoms can be based on any objective or subjective parameter; including, e.g., the result of a physical examination. In embodiments, a treatment alters the natural state of a subject.

As used herein, the term “subject” refers to animals such as mammals. Suitable examples of mammals include, but are not limited to, primates (e.g., humans), cows, sheep, goats, horses, dogs, cats, rabbits, rats, mice, and the like. In certain embodiments, the subject is a human.

As used herein, the terms “therapeutically effective amount” or “therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

### DETAILED DESCRIPTION OF THE INVENTION

In embodiments, the present disclosure provides pharmaceutically acceptable stable formulations with excellent drug loading capacity, reproducibility, and biocompatibility. In embodiments, intratracheal nCMT-3 administration attenuates LPS-induced ALI by attenuating MMP activation, sTREM-1 release and NLRP3 inflammasome activation.

In embodiments, the present disclosure includes a nanocarrier composition, including: one or more telodendrimers suitable for binding to one or more tetracyclines; and one or more tetracyclines have substantially no antibacterial activity. In embodiments, the tetracycline is 6-demethyl-6-deoxy-4-de(dimethylamino)tetracycline (CMT-3), or a derivative thereof. In embodiments, the tetracycline is physically bonded to, or has an affinity to the one or more telodendrimers of the present disclosure.

In embodiments, suitable telodendrimers for use in accordance with the present disclosure are functional segregated telodendrimers having, for example, two or three functional segments. In embodiments, the telodendrimers can have one or more crosslinking groups (e.g., reversible photocrosslinking groups). In embodiments, the telodendrimers of the present disclosure may have a PEG groups. Without intending to be bound by any particular theory, it is considered that the PEG layer serves as a stealth hydrophilic shell to stabilize the nanoparticle and to avoid systemic clearance by the reticuloendothelial system (RES); the intermediate layer contains for example, optional crosslinkable functional group(s), amphiphilic oligo-cholic acid, riboflavin, or chlorogenic acid and can further stabilize nanoparticle and cage drug molecules in the core of nanoparticle; the interior layer contains drug-binding building blocks, such as vitamins (α-tocopherol, riboflavin, folic acid, retinoic acid, etc.) functional lipids (ceramide), chemical extracts (rhein, coumarin, curcurmine, etc) from herbal medicine to increase the affinity to drug molecules.

In embodiments, the present disclosure provides telodendrimers having three functional segments. In an embodiment, the telodendrimer is a tri-block telodendrimer system with segregated functional regions.

In some embodiments, the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof. In embodiments, PEG^{5k}CA₄Ve₄ is characterized by the formula:

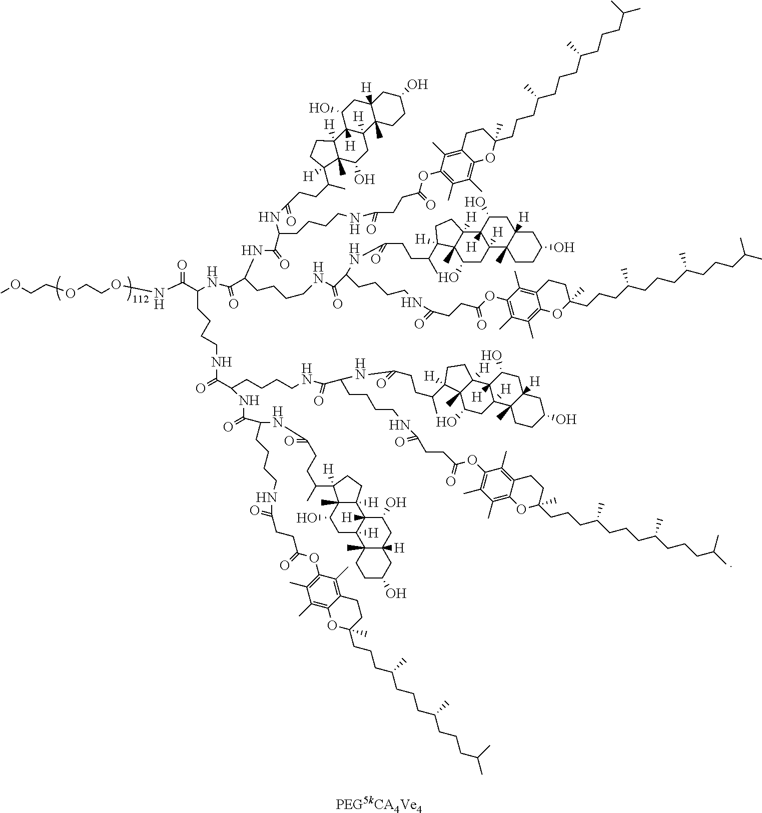

In embodiments, PEG^{5k}CA₄Ura₄ is characterized by the formula:

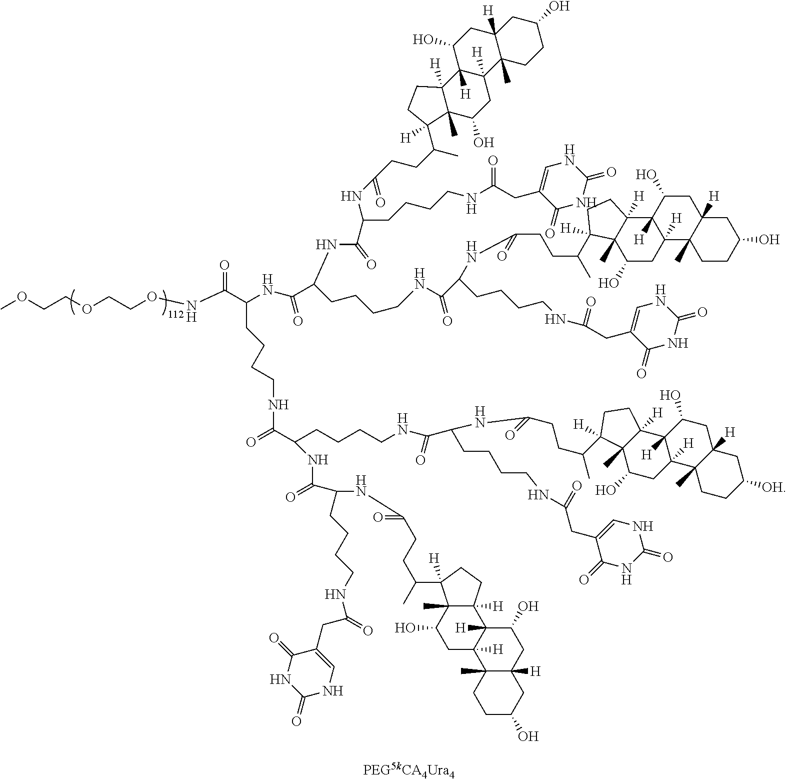

In embodiments, PEG^{nk}CA₄-L-VE₄ is characterized by the formula:

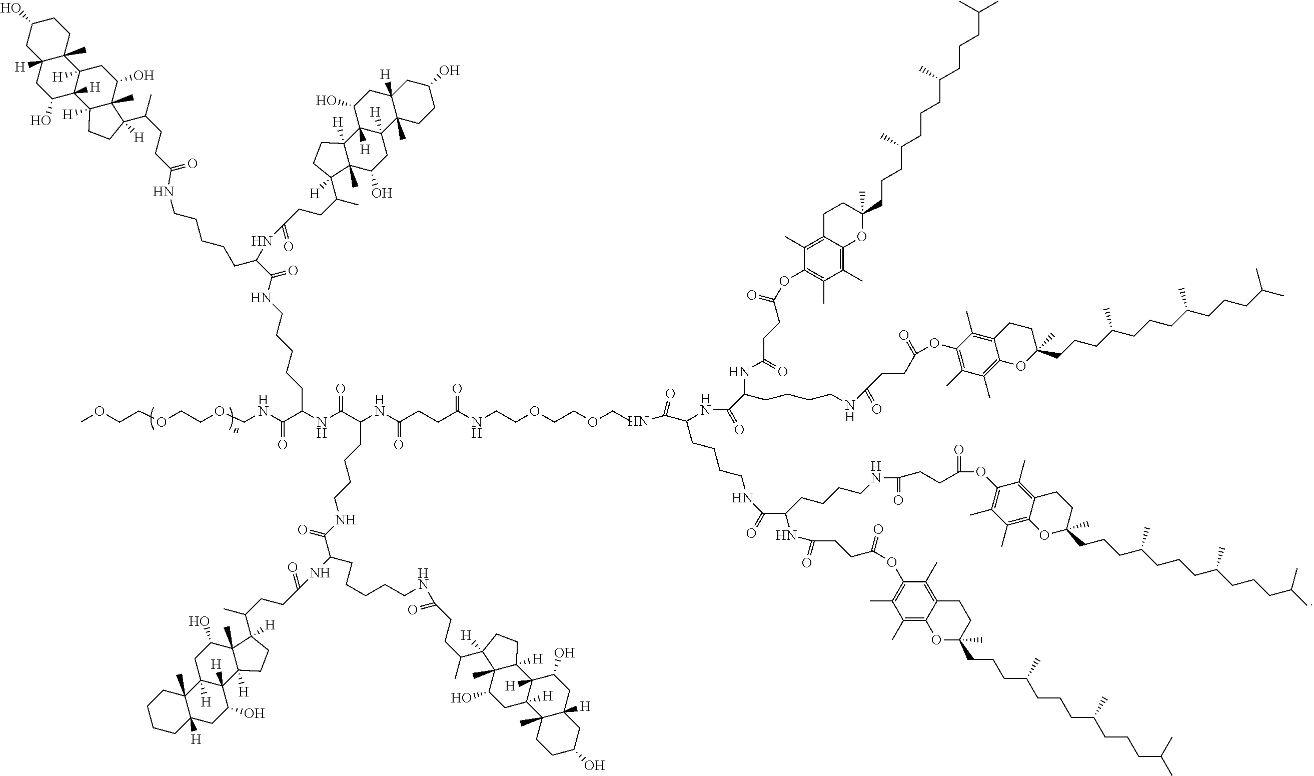

In embodiments, the one or more telodendrimers include one or more of a PEG^{nk} moiety such as PEG^{5k}, a 2 layer poly-lysine moiety, a 3-layer poly-lysine moiety, a CA or cholic acid moiety, a VE or vitamin E moiety, a URA moiety, or combinations thereof. In some embodiments, the one or more telodendrimers include a PEG moiety, wherein the PEG is characterized as PEG^{nk}. In embodiments, the “nk” refers PEG having a molecular weight in the amount of 2-40 kDa, such as 5 kDA. In embodiments, n is a number or integer between 2 and 40. In embodiments, k refers to kilodaltons, or a kDa unit. In some embodiments, the one or more telodendrimers include a two layered poly-lysine moiety, a three-layered poly-lysine moiety, a CA (cholic acid) moiety, a vitamin E (VE) moiety, a URA moiety, a uracil-5-ylacetic acid moiety, a (2,4-dioxide-1,2,3,4-tetrahydro-5-pyrimidinyl) acetic acid moiety, or combinations thereof. In some embodiments, the telodendrimer composition suitable for use herein is characterized by self-assembly drug loading.

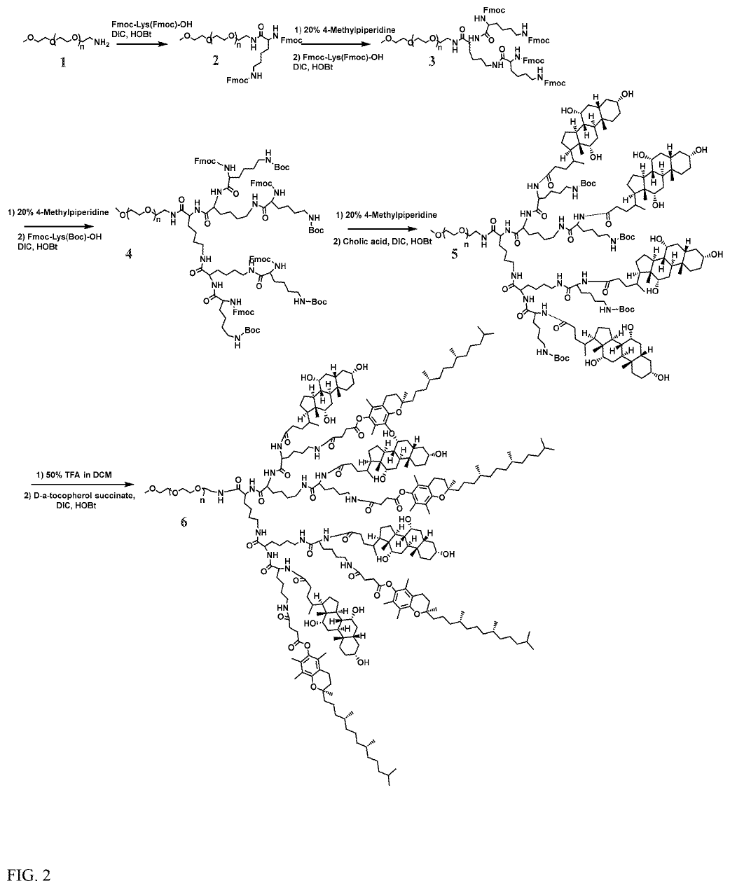

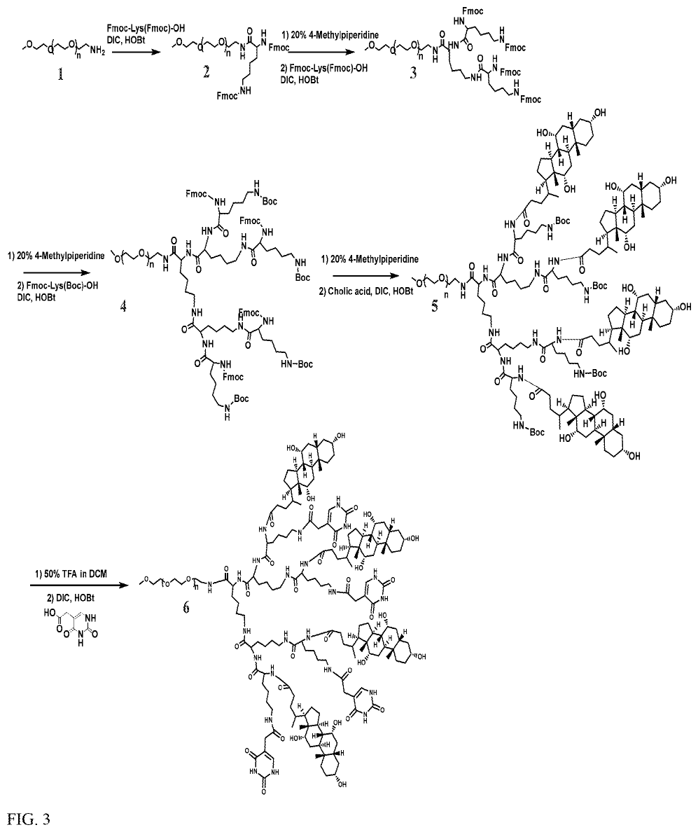

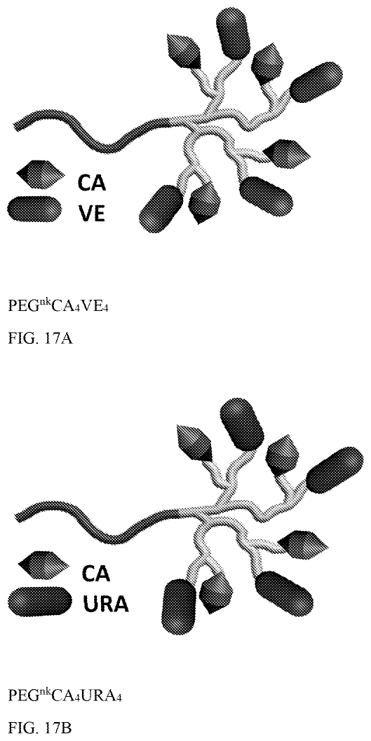

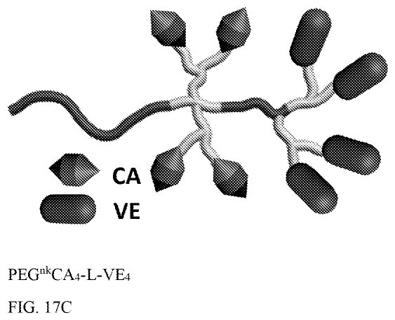

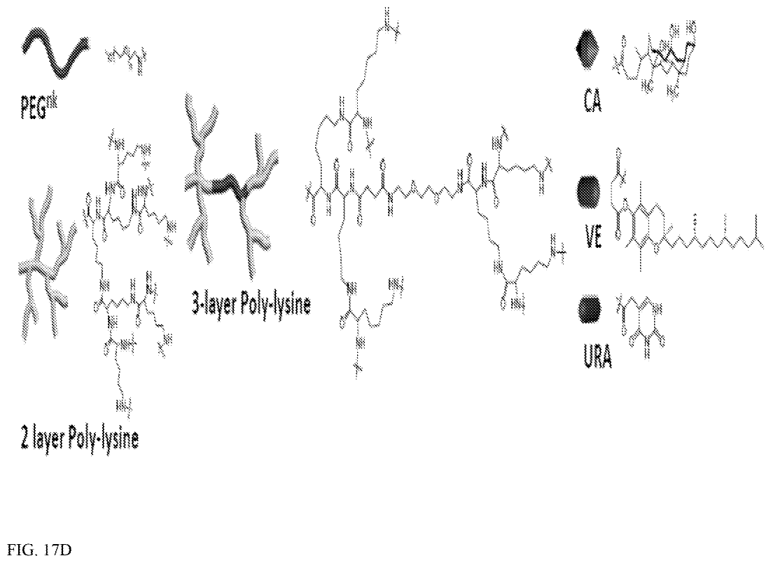

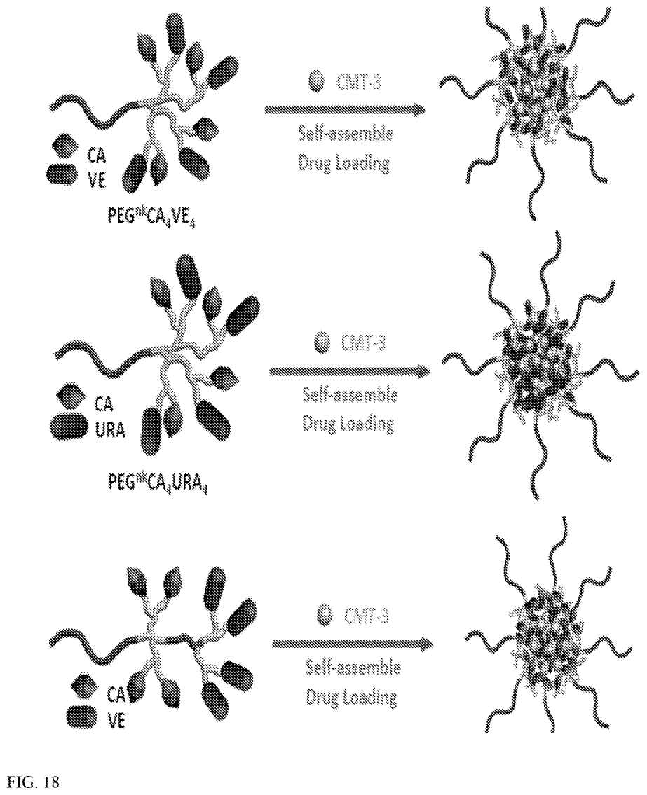

In embodiments, the telodendrimers of the present disclosure can be synthesized via peptide chemistry, which can control the chemical structure and the architecture of the telodendrimers. Efficient stepwise peptide chemistry allows for reproducibility and scaling up for clinical development. FIGS. 2-4 depict suitable schemes, or a synthetic route embodiment of the present disclosure for the formation of PEG^{nk}CA₄VE₄, wherein n=5, PEG^{nk}CA₄URA₄, wherein n=5, and PEG^{nk}CA₄-L-VE₄, respectively. In addition, given their structure, the telodendrimers can self-assemble into micelle nanoparticles with controlled and tunable properties, such as particle size, drug loading capacity and stability. (See e.g., FIGS. 17A-17D and FIG. 18). In embodiments, cholic acid is a facial amphiphilic biomolecule suitable for use herein. In embodiments, as a core-forming building block, cholic acid can play a role in stabilizing nanoparticle and the drug molecules loaded in the nanoparticles. In embodiments, drug-binding bioactive and biocompatible molecules can be introduced into telodendrimer in the core of the micelle to improve the drug loading capacity and stability.

With the aid of computational approaches, a number of natural bioactive compounds for design and synthesis of telodendrimers with segregated functional layers for efficient delivery of specific drug molecules were examined, (e.g., antibiotics such as CMT-3). The loading capacity and stability of these nanotherapeutics have been significantly improved via engineering the topology of the telodendrimers. In embodiments, the particle sizes of these nanoformulations can be within the optimal range of 10-30 nm for efficient in vivo tissue targeting.

### Nanocarriers

In embodiments, the present disclosure provides nanocarriers including the telodendrimers of the present disclosure, alone or in combination with CMT-3, or a pharmaceutically acceptable salt thereof, or CMT-3 analogues, derivatives thereof and/or pharmaceutically acceptable salts thereof. In embodiments, the nanocarriers are nontoxic in cell culture and the drug-loaded nanoformulations exhibit excelled potency in vitro, and better anti-inflammatory effects in vivo, due to the tissue targeted drug delivery. In embodiments, the resulting nanocarriers exhibit superior drug loading capacity and stability. The side toxicities of the anti-inflammatory drugs were significantly reduced via nanoformulation. The optimized nanoparticle is able to target delivery of the payload anti-inflammatory drugs to the tissue site in need thereof, such as lung tissue. As a result, custom designed telodendrimer nanotherapeutics significantly improve the anti-inflammatory effects in vivo.

In embodiments, the telodendrimers of the present disclosure can aggregate to form nanocarriers with a hydrophobic core and a hydrophilic exterior. In an embodiment, a plurality of telodendrimers aggregate to form nanocarriers with a hydrophobic core and a hydrophilic exterior. In an embodiment, the disclosure provides a nanocarrier having an interior and an exterior, the nanocarrier including a plurality of the telodendrimer conjugates of the disclosure, wherein each compound self-assembles in an aqueous solvent to form the nanocarrier such that a hydrophobic pocket is formed in the interior of the nanocarrier, and wherein the hydrophilic segment (e.g., PEG) of each compound self-assembles on the exterior of the nanocarrier. (See e.g., FIG. 18).

In embodiments, the nanocarrier includes a hydrophobic drug or an imaging agent, such that the hydrophobic drug or imaging agent is sequestered in the hydrophobic pocket of the nanocarrier. Hydrophobic drugs useful in the nanocarrier of the present disclosure include any drug having low water solubility. In some embodiments, the hydrophobic drug in the nanocarrier can be CMT-3 or an analogue thereof. However, other drugs may be included such as bortezomib, gambogic acid, oridonin, norcantharidin, triptolide, paclitaxel, SN38, amphotericin B, camptothecin, etoposide and doxorubicin, docetaxel, daunorubicin, VP 16, prednisone, methotrexate, cisplatin, carboplatin, oxapaltin, dexamethasone, vincristine, vinblastine, temsirolimus, and carmusine.

In some embodiments, the nanocarrier includes at least one monomer unit that is optionally linked to an optical probe, a radionuclide, a paramagnetic agent, a metal chelate or a drug. The drug can be a variety of hydrophilic or hydrophobic drugs, and is not limited to the hydrophobic drugs that are sequestered in the interior of the nanocarriers of the present disclosure.

Drugs that can be sequestered in the nanocarriers or linked to the conjugates of the present disclosure include, but are not limited to, CMT-3, analogues and derivatives thereof.

In some embodiments, the tetracycline compound is modified chemically to reduce or eliminate its antimicrobial properties. Chemically modified nonantimicrobial tetracyclines (CMT's) include, for example, 4-de(dimethylamino)tetracycline (CMT-1), tetracyclinonitrile (CMT-2), 6-demethyl-6-deoxy-4-de(dimethylamino)tetracycline (CMT-3), 7-chloro-4-de(dimethylamino)tetracycline (CMT-4), tetracycline pyrazole (CMT-5), 4-hydroxy-4-de(dimethylamino)tetracycline (CMT-6), 4-de(dimethylamino-12.alpha.-deoxytetracycline (CMT-7), 6-deoxy-5.alpha.-hydroxy-4-de(dimethylamino)tetracycline (CMT-8), 4-de(dimethylamino)-12α-deoxyanhydrotetracycline (CMT-9), 4-de(dimethylamino)minocycline (CMT-10). In embodiments, the chemically modified tetracyclines can be made by methods known in the art. See, for example, Mitscher, L. A., The Chemistry of the Tetracycline Antibiotics, Marcel Dekker, New York (1978), Ch. 6, and U.S. Pat. Nos. 4,704,383 and 5,532,227.

In embodiments, the tetracycline compound includes the following chemical formula:

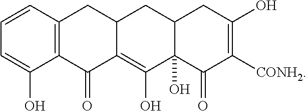 [SMILES: NC(=O)C1=C(O)CC2CC3Cc4cccc(O)c4C(=O)C3=C(O)[C@]2(O)C1=O]

In embodiments, the amount of the tetracycline compound administered is any amount effective for reducing or inhibiting neutrophil accumulation in the lungs. Nonantimicrobial tetracycline derivatives can be used at higher levels than antimicrobial tetracyclines, while avoiding certain disadvantages, such as the indiscriminate killing of beneficial microbes which often accompanies the use of antimicrobial or antibacterial amounts of such compounds.

In some embodiments, the present disclosure may include a second drug (alone or incombination with a first drug) such as cytostatic agents, cytotoxic agents (such as for example, but not limited to, DNA interactive agents (such as cisplatin or doxorubicin)); taxanes (e.g., taxotere, taxol); topoisomerase II inhibitors (such as etoposide); topoisomerase I inhibitors (such as irinotecan (or CPT-11), camptostar, or topotecan); tubulin interacting agents (such as paclitaxel, docetaxel or the epothilones); hormonal agents (such as tamoxifen); thymidilate synthase inhibitors (such as 5-fluorouracil); anti-metabolites (such as methotrexate); alkylating agents (such as temozolomide (TEMODAR™ brand drug from Schering-Plough Corporation, Kenilworth, N.J.), cyclophosphamide); aromatase combinations; ara-C, adriamycin, cytoxan, and gemcitabine. Other drugs useful in the nanocarrier of the present disclosure include but are not limited to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ brand drug from Sanofi-Synthelabo Pharmaceuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17.alpha.-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, or Hexamethylmelamine. Prodrug forms are also useful in the disclosure.

In embodiments, the nanocarriers may further include one or more imaging agents such as paramagnetic agents, optical probes and radionuclides. Paramagnetic agents include iron particles, such as iron nanoparticles that are sequestered in the hydrophobic pocket of the nanocarrier.

In some embodiments, the present disclosure includes a nanocarrier composition, including: one or more telodendrimers suitable for binding to a tetracycline, wherein the tetracycline is 6-demethyl-6-deoxy-4-de(dimethylamino)tetracycline (CMT-3), or a derivative thereof, and wherein the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof.

In embodiments, a nanocarrier composition, includes: one or more telodendrimers suitable for binding to one or more tetracyclines; and one or more tetracyclines have substantially no antibacterial activity. In embodiments, the tetracycline is 6-demethyl-6-deoxy-4-de(dimethylamino)tetracycline (CMT-3), or a derivative thereof. In embodiments, the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof. In embodiments, the one or more telodendrimers include the formula:

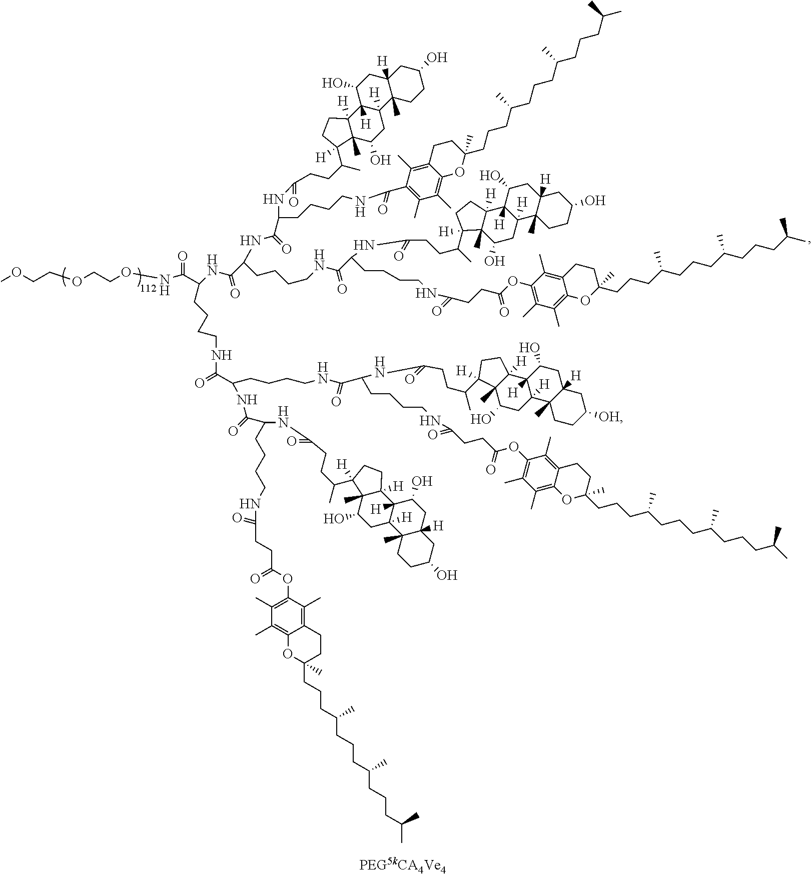

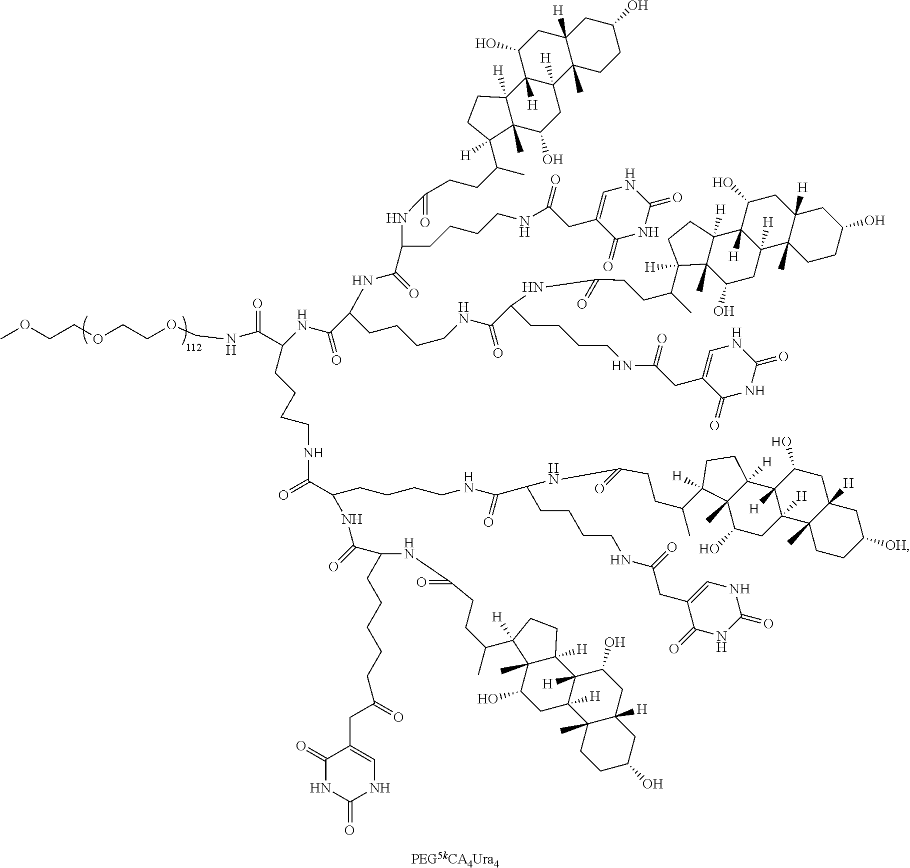

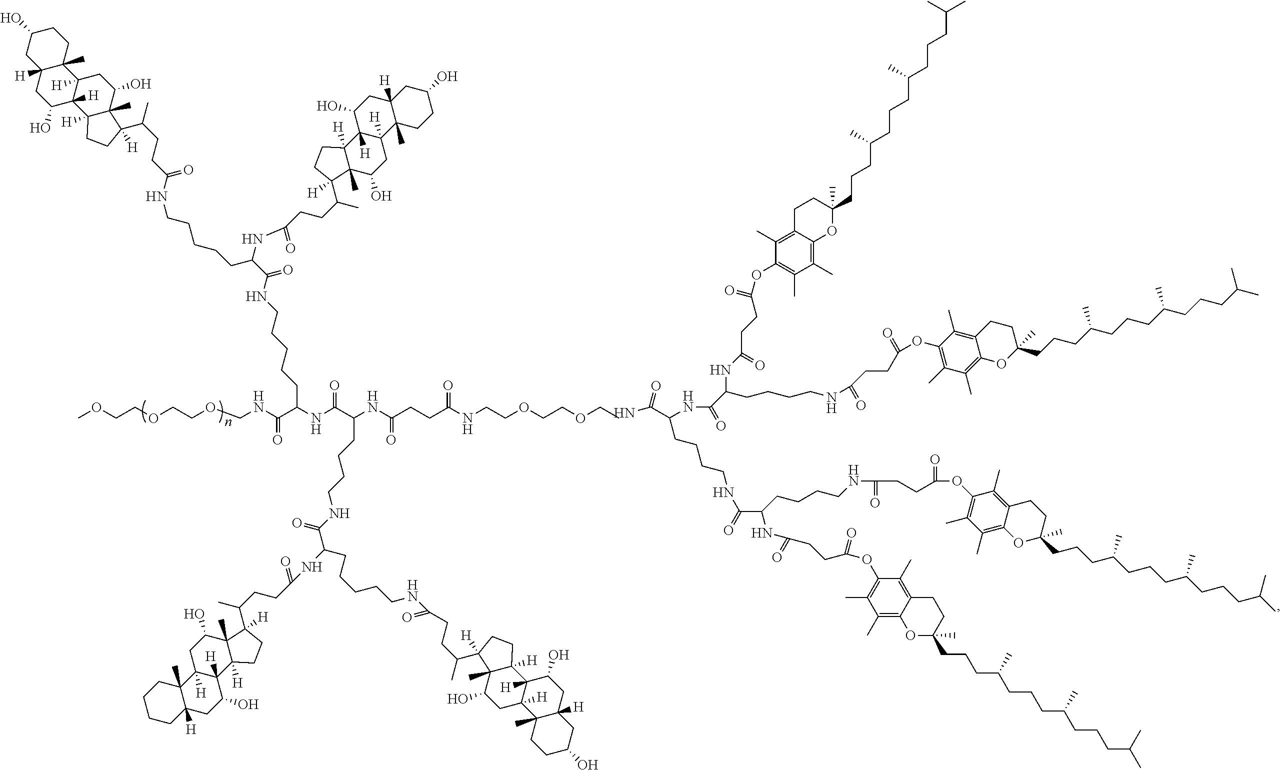

or combinations thereof.

In embodiments, the one or more telodendrimers include one or more of a PEG^{nk} moiety, such as wherein n is a number or integer in the amount of 1-40, or wherein nk is 1-40 kDa, a 2 layer poly-lysine moiety, a 3-layer poly-lysine moiety, a CA moiety, a VE moiety, a URA moiety, or combinations thereof. In embodiments, the nanocarrier composition is characterized by self-assembly drug loading.

### Method of Treating

The nanocarriers of the present disclosure can be used to treat lung disease requiring the administration of a drug, such as by sequestering a hydrophobic drug in the interior of the nanocarrier, or by covalent attachment of a drug to a conjugate of the nanocarrier. The nanocarriers can also be used for imaging, by sequestering an imaging agent in the interior of the nanocarrier, or by attaching the imaging agent to a conjugate of the nanocarrier.

In embodiments, the nanocarriers of the present disclosure can be used to treat inflammatory disease requiring the administration of a drug, such as by sequestering a drug such as CMT-3 in the interior of the nanocarrier, and/or atop the nanocarrier, or by physical or covalent attachment of a drug to a conjugate of the nanocarrier or to the nanocarrier.

In some embodiments, the present disclosure provides a method of treating a disease, including administering to a subject in need of such treatment a therapeutically effective amount of a nanocarrier of the present disclosure, where the nanocarrier includes a drug such as CMT-3. In embodiments, the drug can be covalently attached to a conjugate of the nanocarrier. In embodiments, the drug can be non-covalently attached to a conjugate of the nanocarrier, such as by physical binding. In some embodiments, the drug is a hydrophobic drug sequestered in the interior of the nanocarrier. In some embodiments, the nanocarrier also includes an imaging agent. In embodiments, the imaging agent can be a covalently attached to a conjugate of the nanocarrier, or the imaging agent can be sequestered in the interior of the nanocarrier. In some other embodiments, both a hydrophobic drug and an imaging agent are sequestered in the interior of the nanocarrier. In still other embodiments, both a drug and an imaging agent are covalently linked to a conjugate or conjugates of the nanocarrier. In yet other embodiments, the nanocarrier can also include a radionuclide.

In some embodiments, the present disclosure provides a method for treating inflammation or inflammatory disease in a mammal, the method including: administering a nanocarrier composition to a mammal in need thereof in a therapeutically effective amount, wherein the nanocarrier composition includes one or more telodendrimers suitable for binding to one or more tetracyclines, wherein the one or more tetracyclines have substantially no antibacterial activity. In embodiments, the tetracycline is 6-demethyl-6-deoxy-4-de(dimethylamino)tetracycline (CMT-3), or a derivative or analogue thereof. In embodiments, the one or more tetracyclines are present in a therapeutically effective amount. In some embodiments, the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof.

In some embodiments, the present disclosure includes a method for preventing, alleviating, or treating acute lung injury in a mammal, the method including: administering a nanocarrier composition to a mammal in need thereof in a therapeutically effective amount, wherein the nanocarrier composition includes one or more telodendrimers suitable for binding to one or more tetracyclines, wherein the one or more tetracyclines have substantially no antibacterial activity. In embodiments, the tetracycline is 6-demethyl-6-deoxy-4-de(dimethylamino)tetracycline (CMT-3), or an analogue, or derivative thereof. In some embodiments, the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof. For example, in embodiments, the one or more telodendrimers include the formula:

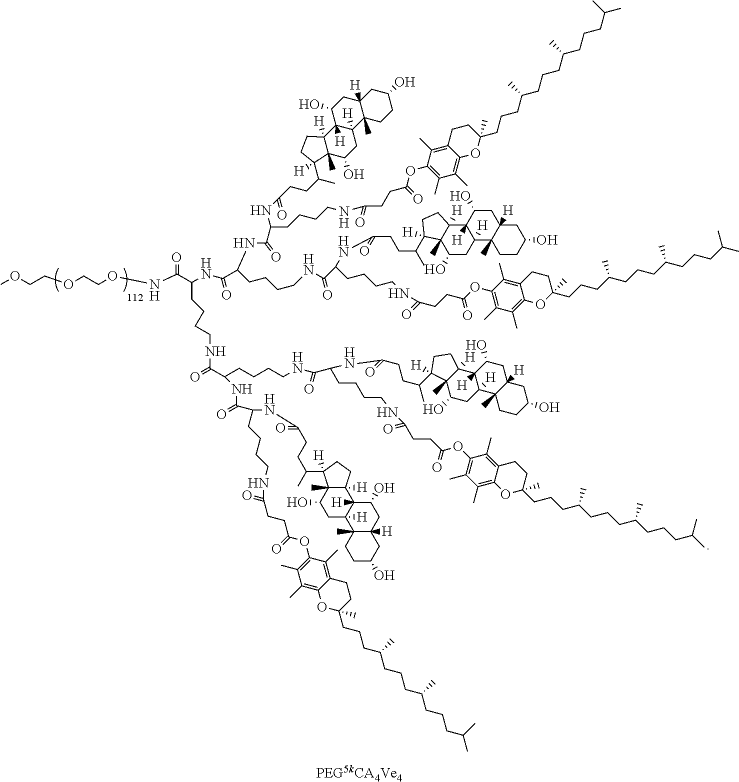

In another embodiment, the one or more telodendrimers comprise the formula:

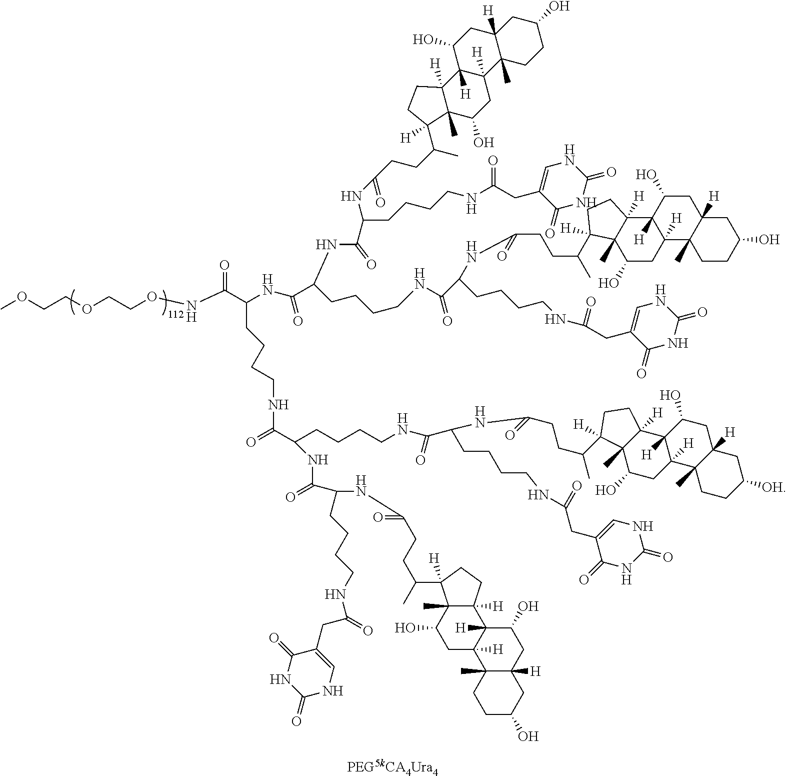

In another embodiments, the one or more telodendrimers comprise the formula:

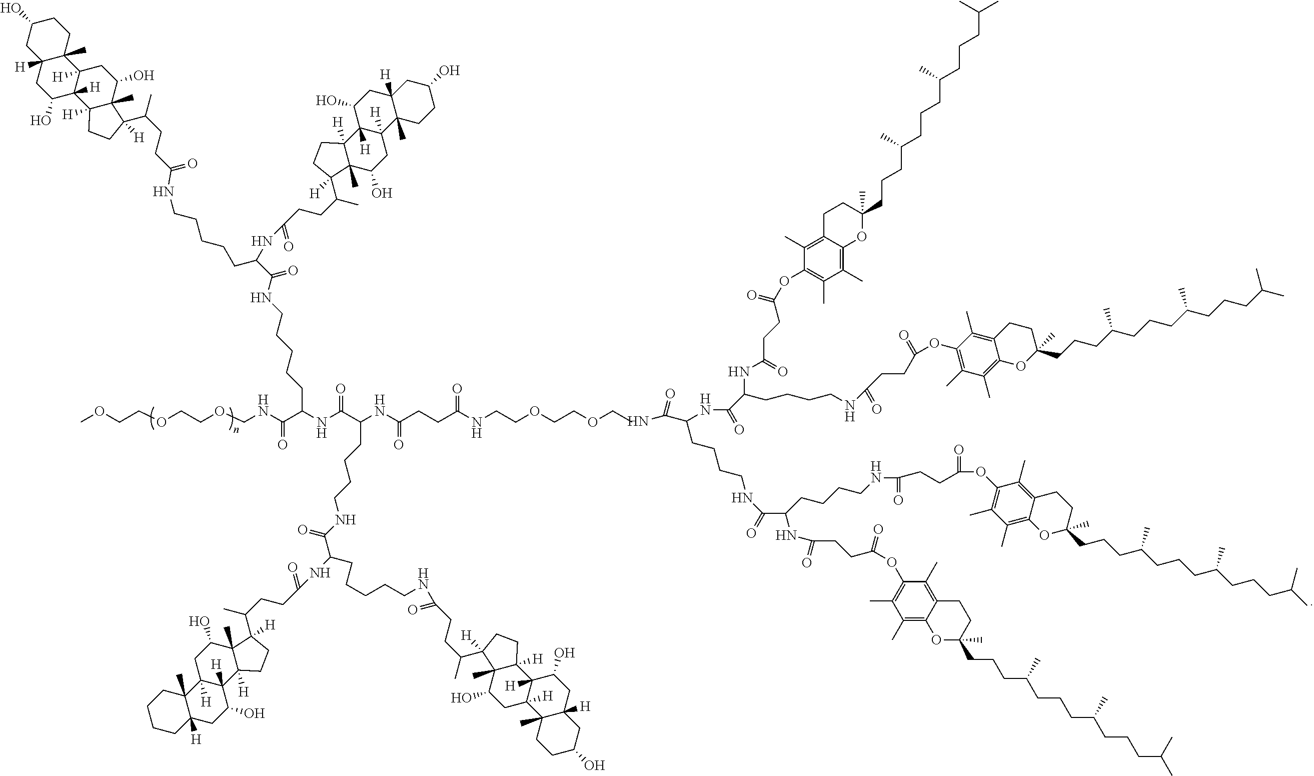

In some embodiments, the one or more telodendrimers include one or more of a PEG^{nk} moiety, wherein n=1-40, 1-20, 1-5, or about 5, or 5, a two-layer poly-lysine moiety, a three-layer poly-lysine moiety, a CA moiety, a VE moiety, a URA moiety, or combinations thereof. In some embodiments, the composition is characterized by self-assembly drug loading.

In addition, the nanocarriers of the present disclosure are useful for the treatment of infection by pathogens such as viruses, bacteria, fungi, and parasites. Other diseases can be treated using the nanocarriers of the present disclosure.

### Formulations

In embodiments, the nanocarriers of the present disclosure can be formulated in a variety of different manners known to one of skill in the art. Pharmaceutically acceptable carriers are determined in part by the particular composition being administered, as well as by the particular method used to administer the composition. Accordingly, there are a wide variety of suitable formulations of pharmaceutical compositions of the present disclosure (see, e.g., Remington's Pharmaceutical Sciences, 20^{th} ed., 2003, supra). Effective formulations include oral and nasal formulations, formulations for parenteral administration, and compositions formulated for with extended release.

Formulations suitable for oral administration can consist of (a) liquid solutions, such as an effective amount of a compound of the present disclosure suspended in diluents, such as water, saline or PEG 400; (b) capsules, sachets, depots or tablets, each containing a predetermined amount of the active ingredient, as liquids, solids, granules or gelatin; (c) suspensions in an appropriate liquid; (d) suitable emulsions; and (e) patches. The liquid solutions described above can be sterile solutions. The pharmaceutical forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Preferred pharmaceutical preparations can deliver the compounds of the disclosure in a sustained release formulation.

Pharmaceutical preparations useful in the present disclosure also include extended-release formulations. In some embodiments, extended-release formulations useful in the present disclosure are described in U.S. Pat. No. 6,699,508, which can be prepared according to U.S. Pat. No. 7,125,567, both patents incorporated herein by reference.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

In practicing the methods of the present disclosure, the pharmaceutical compositions can be used alone, or in combination with other therapeutic or diagnostic agents.

In embodiments, the nanocarriers of the present disclosure are pharmaceutically acceptable, and/or suitable for use in a pulmonary inhalation. Accordingly, in embodiments, administration of pharmaceutical preparations of the present disclosure is suitable by inhalation so that they reach the lungs and in particular embodiments the alveolar regions of the lung. In embodiment, the nanocarriers are provided in formulations suitable for aerosolized administration such as through a nebulizer or dry powder form. In embodiments, the nanocarriers can be administered in a known aerosol deliver device such as those described in U.S. Patent Publication No. 20160331030, WO2002083220, U.S. Pat. Nos. 9,358,352, 8,485,180, 10,159,644 (all of which are herein incorporated by reference).

In embodiments, the present disclosure includes an inhalable composition including: one or more telodendrimers suitable for binding to one or more tetracyclines; and one or more tetracyclines having substantially no antibacterial activity. In embodiments, the one or more one or more telodendrimers and one or more tetracyclines are disposed within a soluble medium suitable for aerosolized administration such as through a nebulizer. In embodiments, the one or more one or more telodendrimers and one or more tetracyclines are disposed within a dry powder composition. In embodiments, a dry powder delivery system includes a breath powered, dry powder, single use inhaler including a dry powder composition including one or more telodendrimers suitable for binding to one or more tetracyclines; and one or more tetracyclines having substantially no antibacterial activity. In embodiments, the amount of dry powder in a composition the amount can range from about 0.001 μg to more than 1 mg, or 0.001 μg to 1 mg, or 0.01 μg to 1 mg, or 0.1 μg to 0.1 mg, or 0.1 μg to 0.05 mg, or the like. In some embodiments, the amount of dry powder to be administered to a subject can be, for example, greater than 0.5 mg, greater than 1 mg, greater than 2 mg, greater than 5 mg, greater than 10 mg, greater than 15 mg, greater than 20 mg, greater than 30 mg, or the like. In embodiments, the dry powder compositions includes a therapeutically effective amount or dose or drug such as CMT-3.

In embodiments, the dry powder delivery systems include an inhaler, a unit dose dry powder medicament container, and a powder including the nanocarriers disclosed herein and a drug such as anti-inflammatory tetracycline. In some embodiments the container can be a cartridge that is loaded into the inhaler; in other embodiments the container is integral with the inhaler. In one embodiment, the delivery system for use with the dry powders includes an inhalation system including a high resistance inhaler having air conduits which impart a high resistance to airflow through the conduits for deagglomerating and dispensing the powder. In one embodiment, the inhalation system has a resistance value suitable for dry powder deliver. In certain embodiments, the dry powders can be delivered effectively by inhalation with an inhalation system wherein the peak inhalation pressure differential can range from about 2 to about 20 kPa, which can produce resultant peak flow rates of about between 7 and 70 liters per minute. In certain embodiments, the inhalation system is configured to provide a single dose by discharging powder from the inhaler as a continuous flow, or as one or more pulses of powder delivered to a patient in, for example, less than 5 seconds, or less than 4 seconds, or less than 3 seconds, or less than 2 seconds or less

In an alternate embodiment, dry powder formulations as described herein can include pharmaceutically acceptable carriers and/or excipients ranging from about 0.5% to about 30% by weight, from about 0.9% to about 25% by weight, from about 5% to about 20% by weight, from about 10% to about 15% by weight of the total dry powder composition. Optionally, dry powder compositions can comprise surfactants, or adjuvants.

In some embodiments, the nanocarriers of the present disclosure are disposed within a pharmaceutically acceptable aerosol composition. In embodiments, the aerosol composition may be disposed within a jet nebulizer, pneumatic nebulizer, ultrasonic nebulizer, or electrostatic nebulizer, as is known in the art. In embodiments, a delivery device produces an aerosol, wherein more than 55 wt % of the drug, such as CMT-3 is in the form of particles having a diameter greater than 0.7 microns and less than 5.8 microns. In a preferred embodiment, the aerosolization subassembly includes an AIRLIFE™ MISTY-NEB™ Nebulizer (Allegiance Healthcare, McGaw Park, Illinois, USA). Alternatively, the aerosolized subassembly can be adapted for dry powder drug delivery. That is, the subassembly can be designed to inject or release a dry powder dose into a flowing stream of gas. This can be done by adapting known dry powder inhalers or metered dose inhalers. In one embodiment, the drug reservoir can include a blister or pouch containing the dose, and the blister or pouch can then be ruptured by a mechanical trigger mechanism. In embodiments, a gas flow can then expel the drug from the pouch or blister and disperse the drug for inhalation along with the gas flow. See e.g., WO2003049791 entitled Medical device for inhalation of aerosolized drug with heliox (herein incorporated by reference).

In some embodiments, the one or more one or more telodendrimers and one or more tetracyclines are disposed within a soluble medium suitable for injectable administration such as via intravenous administration. In embodiments, the injectable formulation includes a pharmaceutically acceptable carrier solution such as saline, water, or the like.

### Administration

The nanocarriers of the present disclosure can be administered as frequently as necessary, including hourly, daily, weekly or monthly. The compounds utilized in the pharmaceutical method of the disclosure are administered at the initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of disease diagnosed in a particular patient. The dose administered to a patient, in the context of the present disclosure should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch or pump.

The pharmaceutical compositions can be administered to the patient in a variety of ways, including topically, parenterally, intravenously, intradermally, subcutaneously, intramuscularly, colonically, rectally or intraperitoneally. In embodiments, the pharmaceutical compositions are administered parenterally, topically, intravenously, intramuscularly, subcutaneously, orally, or nasally, such as via inhalation.

In embodiments, in practicing the methods of the present disclosure, the pharmaceutical compositions can be used alone, or in combination with other therapeutic or diagnostic agents. The additional drugs used in the combination protocols of the present disclosure can be administered separately or one or more of the drugs used in the combination protocols can be administered together, such as in an admixture. Where one or more drugs are administered separately, the timing and schedule of administration of each drug can vary. The other therapeutic or diagnostic agents can be administered at the same time as the compounds of the present disclosure, separately or at different times.

The maximal dosage for a subject is the highest dosage which does not cause undesirable or intolerable side effects. For example, the tetracycline compound can be administered in an amount of from about 0.1 mg/kg/day to about 24 mg/kg/day, and from about 2 mg/kg/day to about 18 mg/kg/day. For the purpose of the present disclosure, side effects include clinically significant antimicrobial or antibacterial activity, as well as toxic effects. For example, a dose in excess of about 50 mg/kg/day would likely produce side effects in most mammals, including humans. In any event, the practitioner is guided by skill and knowledge in the field, and the present disclosure includes without limitation dosages which are effective to achieve the described phenomena.

The preferred pharmaceutical composition for use in the method of the disclosure includes a combination of the tetracycline compound in a suitable pharmaceutical vehicle as understood by practitioners in the art.

For the pharmaceutical purposes described above, the tetracycline of the disclosure can be formulated per se in pharmaceutical preparations optionally with known pharmaceutically acceptable adjuvants or carriers. These preparations can be made according to conventional chemical methods and can be administered internally, e.g., orally by tablet or liquid, or by suppository; parenterally, e.g., intravenously, intramuscularly or subcutaneously, as injectable solutions or suspensions; topically or in the form of a spray or aerosol of droplets within the respirable range for inhalation into the lungs and airways. Such aerosols may include vehicles such as pulmonary surfactant preparations which may contribute additional therapeutic efficacy. Time-release or controlled-delivery administration may be employed.

It will be appreciated that the actual amounts of active compound in a specified case will vary according to the particular compositions formulated, the mode of application, and the particular sites and subject being treated. Dosages will be determined using conventional considerations, e.g., by customary comparison of the differential activities of the formulations and of a known agent, e.g., by means of an appropriate conventional pharmacological protocol.

For the purposes of the instant specification, an acute lung injury is considered prevented if the tetracycline leads to a significant inhibition of the injury. As a result of the treatment, a patient would not sustain any injury, or would sustain significantly less injury, than without the treatment. In other words, the patient would have an improved medical condition as a result of the treatment.

In embodiments, the methods of the present disclosure include administration of the tetracyclines any time prior to significant intrapulmonary accumulation of neutrophils in the lung. Thus, the upper limit of this time period is determined by the significant accumulation of neutrophils in the lung. In embodiments, administration of the tetracyclines occurs within 48 hours after trauma, more preferably within 24 hours after trauma, most preferably within 12 hours after trauma and optimally within 6 hours after trauma. Significant intrapulmonary neutrophil accumulation in the lung can be inferred from systemic neutropenia. A white cell count of approximately 4,000 or less white blood cells per microliter of blood is indicative of a neutropenia in which significant neutrophil accumulation in the lung area has occurred.

In embodiments, the present disclosure includes a method of treating inflammation or inflammatory disease including: administering a nanocarrier composition to a mammal in need thereof in a therapeutically effective amount, wherein the nanocarrier composition includes one or more telodendrimers suitable for binding to one or more tetracyclines, wherein the one or more tetracyclines have substantially no antibacterial activity. In embodiments the nanocarrier composition targets, dental, lung, liver, kidney tissues, or combinations thereof. In embodiments, the tetracycline is 6-demethyl-6-deoxy-4-de(dimethylamino)tetracycline (CMT-3), or an analogue or derivative thereof. In embodiments, the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof.

In embodiments, the present disclosure includes a pharmaceutically acceptable composition for pulmonary inhalation, including: one or more nanocarriers including one or more telodendrimers suitable for binding to one or more tetracyclines, wherein the one or more tetracyclines have substantially no antibacterial activity. In embodiments, the composition is suitable for aerosolized delivery to a subject in need thereof. In embodiments, the composition is a dry powder.

In embodiments, the present disclosure includes a pharmaceutically acceptable composition for injectable administration, including: one or more nanocarriers comprising one or more telodendrimers suitable for binding to one or more tetracyclines, wherein the one or more tetracyclines have substantially no antibacterial activity. In embodiments, the composition is a liquid, and includes a therapeutically effective amount of tetracycline. In embodiments, the tetracycline is characterized as an anti-inflammatory.

In embodiments, the present disclosure includes an aerosolized drug delivery system, including: a therapeutically effective amount of CMT-3.

In embodiments, the present disclosure includes an injectable drug delivery system, including: a therapeutically effective amount of CMT-3.

In embodiments, the present disclosure includes a pharmaceutically acceptable composition for pulmonary inhalation, including: one or more tetracyclines, wherein the one or more tetracyclines have substantially no antibacterial activity. In embodiments, the composition is suitable for aerosolized delivery to a subject in need thereof. In embodiments, the composition is a dry powder. In embodiments, the one or more tetracycline is CMT-3.

In embodiments, the present disclosure includes a pharmaceutically acceptable composition for injectable administration, including: one or more tetracyclines having substantially no antibacterial activity. In embodiments, the composition is a liquid, and includes a therapeutically effective amount of tetracycline. In embodiments, the tetracycline is characterized as an anti-inflammatory. In embodiments, the tetracycline is CMT-3.

In embodiments, the pharmaceutically acceptable compositions and treatments described herein are suitable for treating ARDS, CARDS, COVID-19, and the like.

In embodiments, CMT-3 is administered to a subject in need to prevent COVID-19 induced ARDS, improve clinical outcomes, and increase patient survival such as in a severe case of COVID-19 disease. In embodiments, aerosolized CMT-3 is administered in a therapeutically effective amount or dose to a subject in need thereof, such as a subject or patient having or at risk for COVID-19 induced ARDS. In embodiments, the removal of antibiotic properties from CMT-3 minimizes concern for antibiotic resistance during a time where infectious disease control is imperative, and its safety profile is promising for a compromised patient population.

In embodiments, a treatment is provided to reduce systemic inflammation in COVID-19 patients, e.g., one-week 50 mg CMT-3 per day to rapidly suppress inflammation followed by months of treatment with FDA-approved PerioStat (SDD) or doxycycline hyclate.

### Example 1

This example shows examples of syntheses of telodendrimers and supporting data of same of the present disclosure. In embodiments, the functional segregated telodendrimer system (linear dendritic copolymer) of the present disclosure allows for the customized design of the polymer architecture and structures for the efficient delivery of a specific drug with improved loading capacity and stability.

Materials. Methoxypolyethylene glycol amin nk (MeO-PEG^{nk}-NH₂) was purchased from Jenkem. (Fmoc)-Lys(Fmoc)-OH, (Fmoc)-Lys(Boc)-OH, and N-hydroxybenzotriazole (HOBt) were purchased from AnaSpec Inc. Cholic acid (CA), D-α-tocopherol succinate, diisopropyl carbodiimide (DIC), N,N-Dimethylformamide (DMF), dichloromethane (DCM), trifluoroacetic acid (TFA) and other chemical reagents were acquired from Sigma-Aldrich. (2,4-dioxo-1,2,3,4-tetrahydro-5-pyrimidinyl) acetic acid was obtained from ChemBridge Ltd. Dialysis membrane tubing 3500 Dalton molecular weight cut-off (MWCO) was bought from Spectrum Laboratories Inc.

Telodendrimer PEG^{nk}CA₄VE₄ and PEG^{nk}CA₄URA₄ Preparation. As shown in Scheme 1 and 2 below, telodendrimer including four cholic acid and D-α-tocopherol moieties was prepared through solution phase peptide synthesis starting from MeO-PEG^{nk}-NH₂ (1). Fmoc-Lys(Fmoc)-OH (three equiv) was conjugated onto NH₂ group of PEG^{5k} via coupling reagents HOBt and DIC confirming by negative Kaiser test result. PEGylated materials (2) were precipitated by pouring chilled ethyl ether into reaction solution and washed by chilled ethyl ether twice. Fmoc group was de-protected by 20% (v/v) 4-methylpiperidine in DMF, and the product was precipitated and washed three times by chilled ethyl ether. After vacuum drying at room temperature, one (Fmoc)-Lys(Fmoc)-OH (3) and (Fmoc)-Lys(Boc)-OH (4) were coupled sequentially to achieve dendritic polylinsine intermediate with four Fmoc and Boc moieties. To obtain PEG^{5k}CA₄VE₄, four cholic acid groups were conjugated on α-NH₂ of lysine (5) after Fmoc groups removal, while D-α-tocopherol succinate moieties or (2,4-dioxo-1,2,3,4-tetrahydro-5-pyrimidinyl) acetic acid were coupled on the ε-NH₂ (6) after Boc de-protection via 50% TFA (v/v) in DCM.

A route of telodendrimer PEG^{nk}CA₄VE₄ synthesis is shown below in Scheme 1.

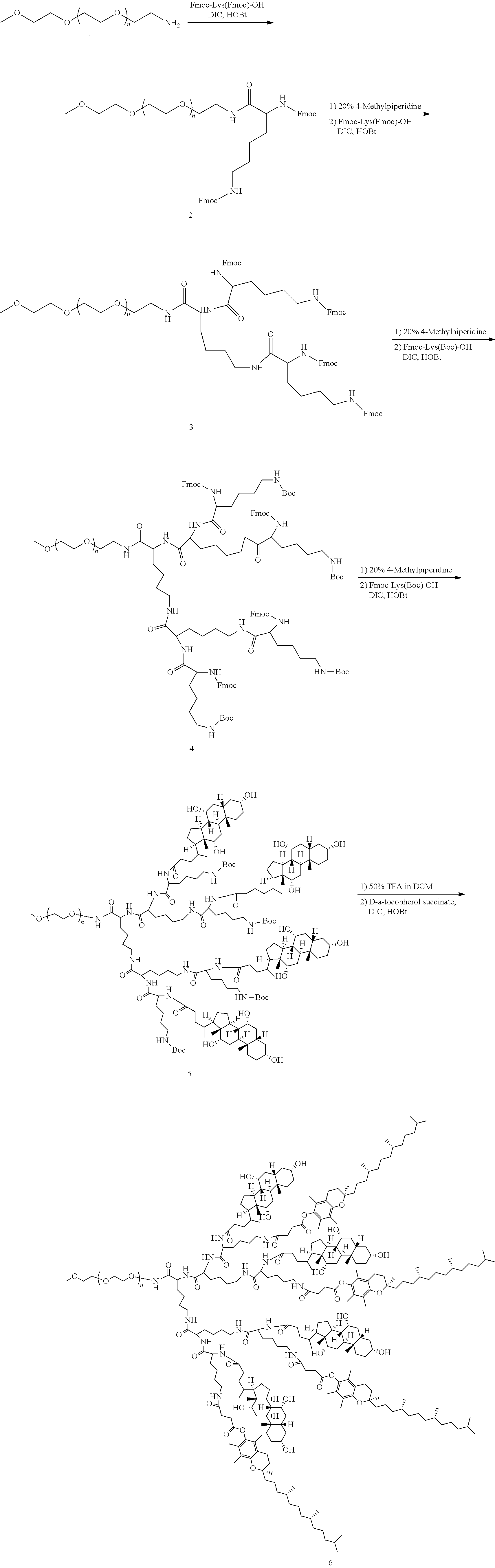

A route of telodendrimer PEG^{nk}CA₄URA₄ synthesis is shown below in scheme 2.

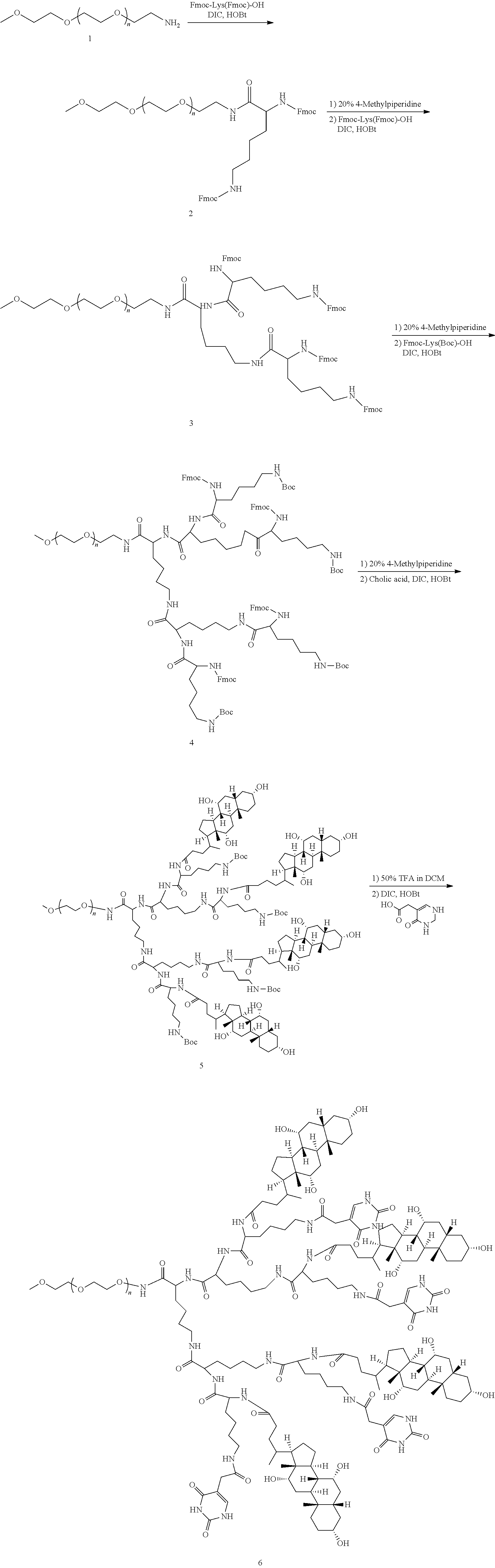

Telodendrimer PEG^{nk}CA₄-L-VE₄ Preparation. As shown in Scheme 3 below, telodendrimer including four cholic acid and D-α-tocopherol moieties was prepared through solution phase peptide synthesis starting from MeO-PEG^{5k}-NH₂ (1). Fmoc-Lys(Boc)-OH (three equiv) was conjugated onto NH₂ group of PEG^{5k} via coupling reagents HOBt and DIC confirming by negative Kaiser test result. PEGylated materials (2) were precipitated by pouring chilled ethyl ether into reaction solution and washed by chilled ethyl ether twice. Fmoc group was de-protected by 20% (v/v) 4-methylpiperidine in DMF, and the product was precipitated and washed three times by chilled ethyl ether. After vacuum drying at room temperature, one (Fmoc)-Lys(Boc)-OH (3), PEG⁴⁷⁰Linker-Fmoc (4), and two couplings of Fmoc-Lys(Fmoc)-OH (5,6) were coupled sequentially to achieve dendritic polylinsine intermediate with four Fmoc moieties. After removal of four Fmoc groups, the polymer was coupled D-α-tocopherola succinate moieties (7). Then Fmoc-Lys(Fmoc)-OH (8) was coupled to the amino groups of the proximal lysines between PEG and PEG linker upon removal of Boc groups with 50% (v/v) DCM and TFA. To obtain PEG^{5k}CA₄-L-VE₄ (9), four cholic acid groups were conjugated on α-NH₂ of lysine after Fmoc groups removal.

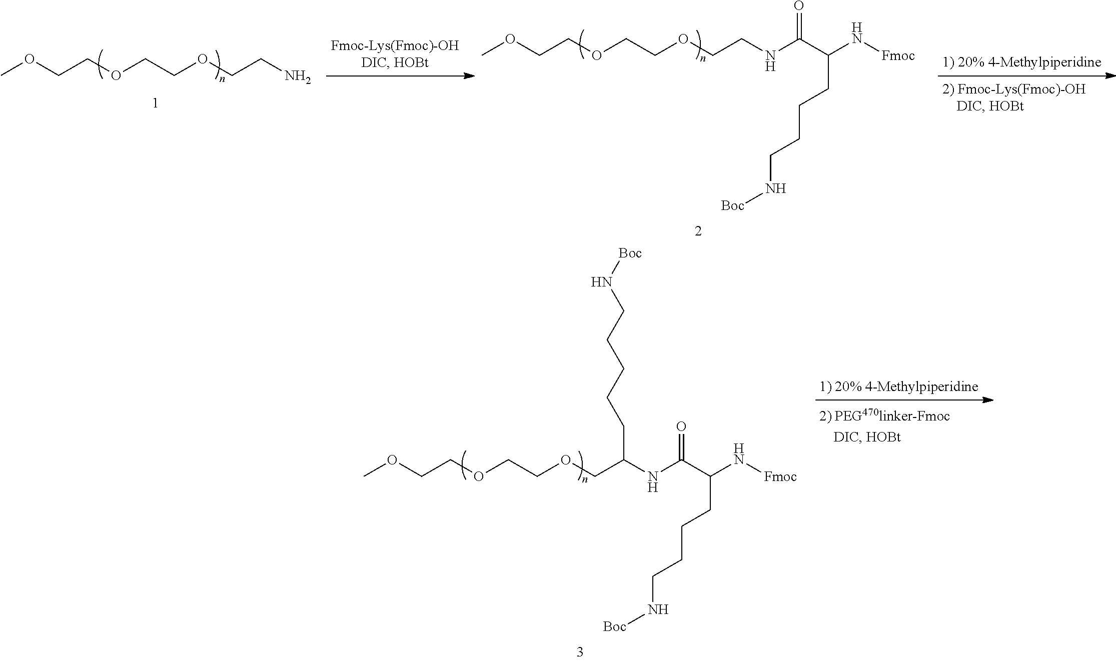

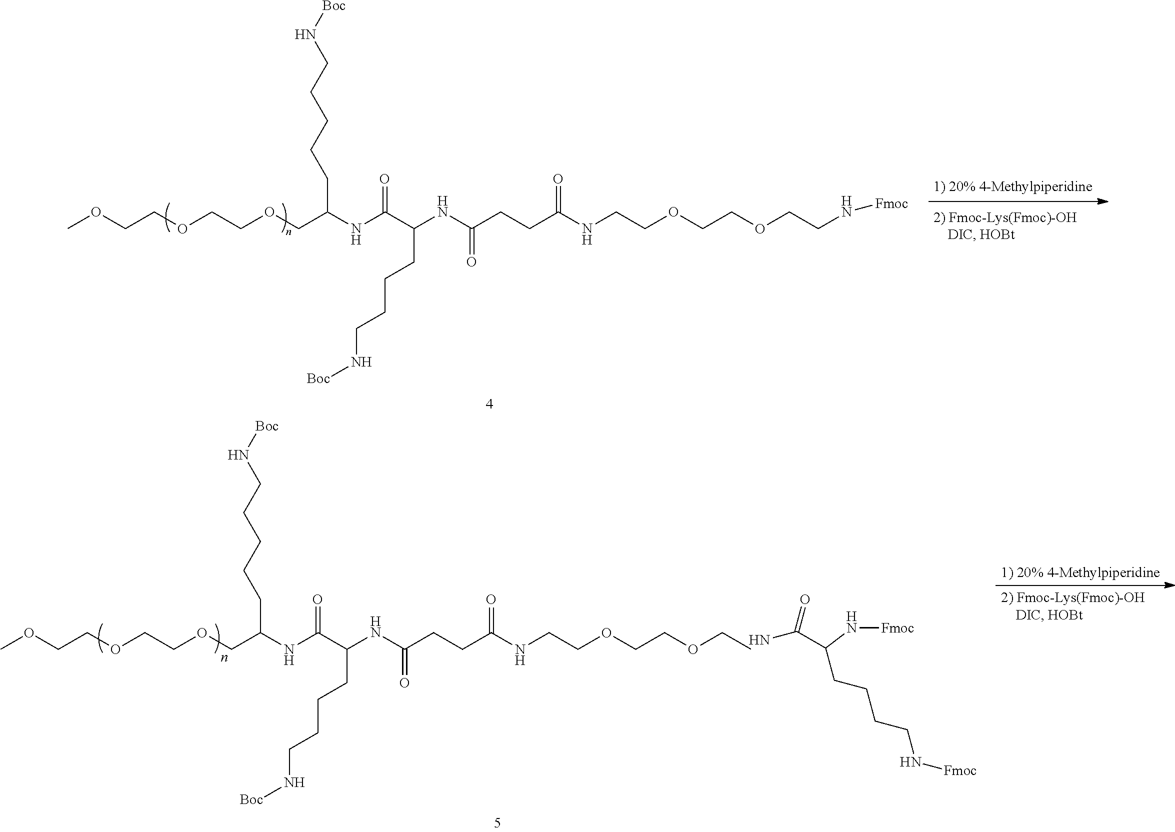

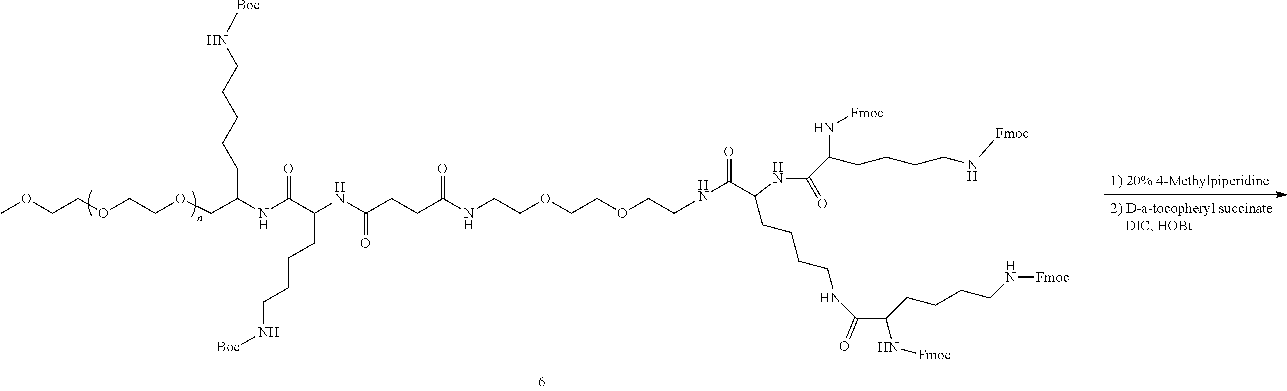

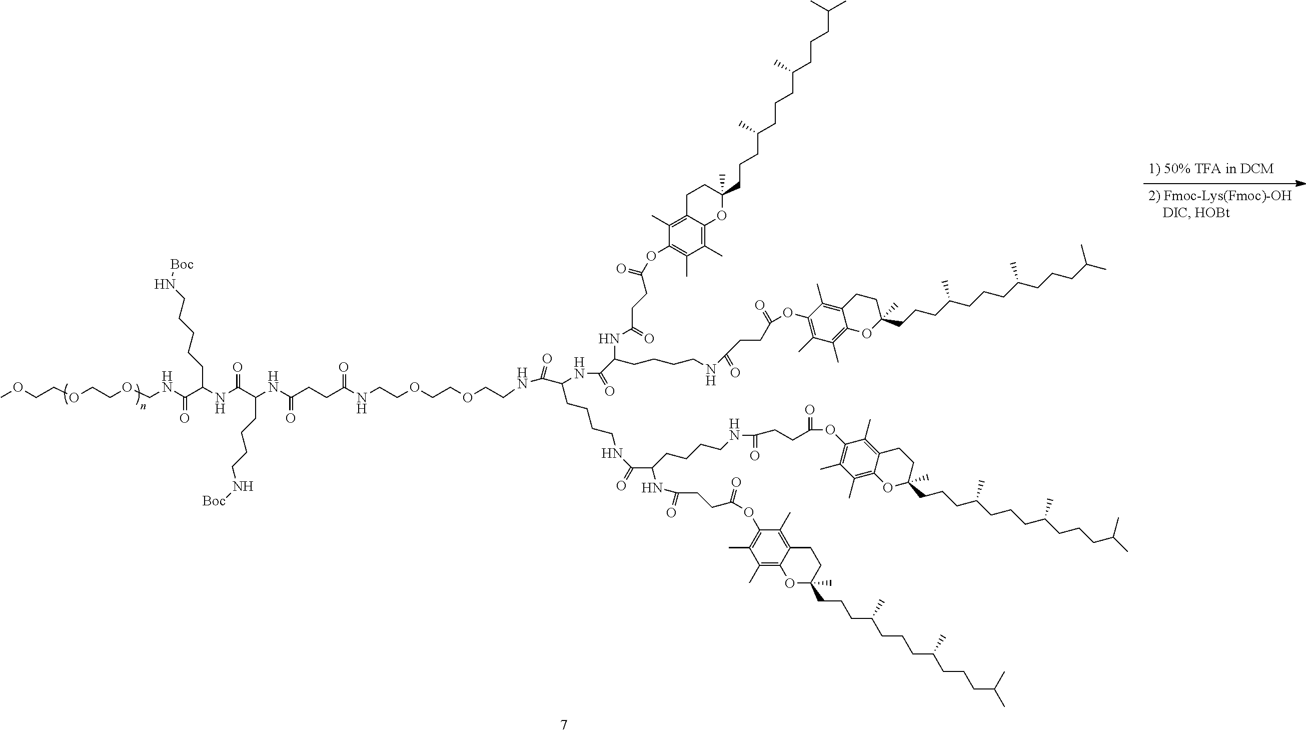

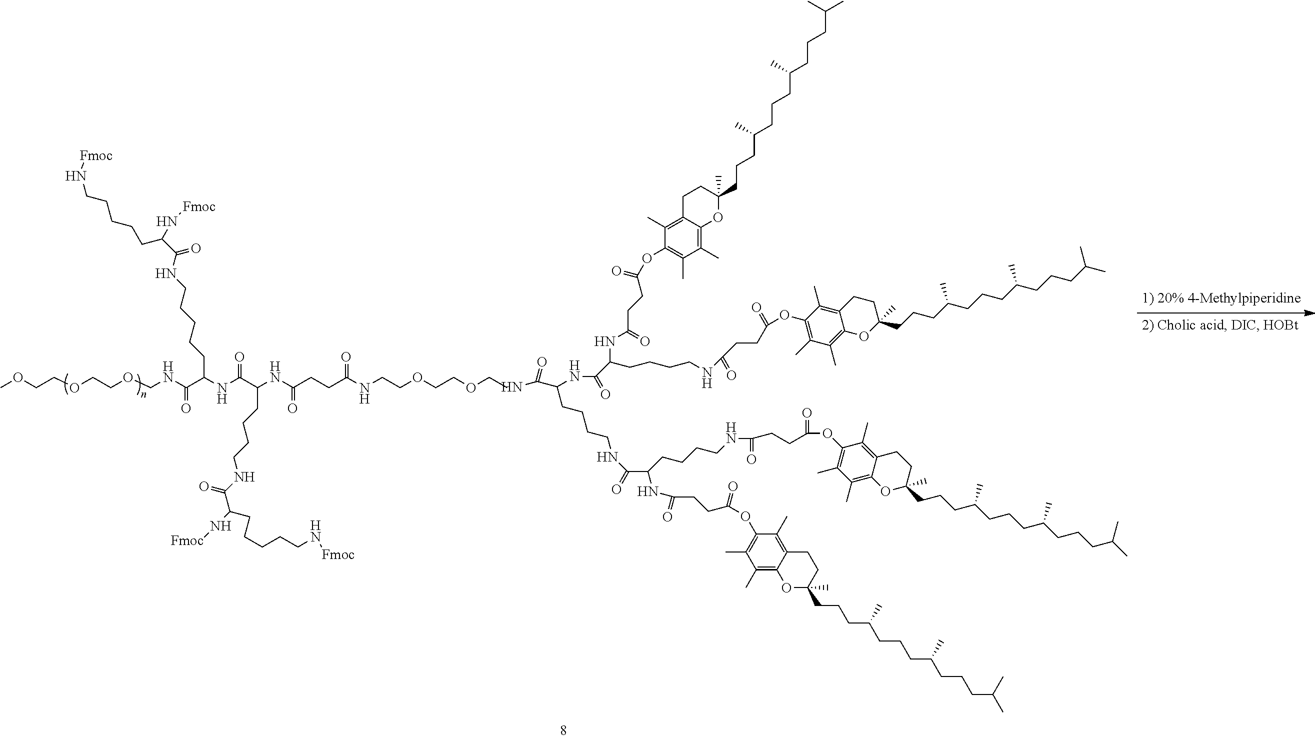

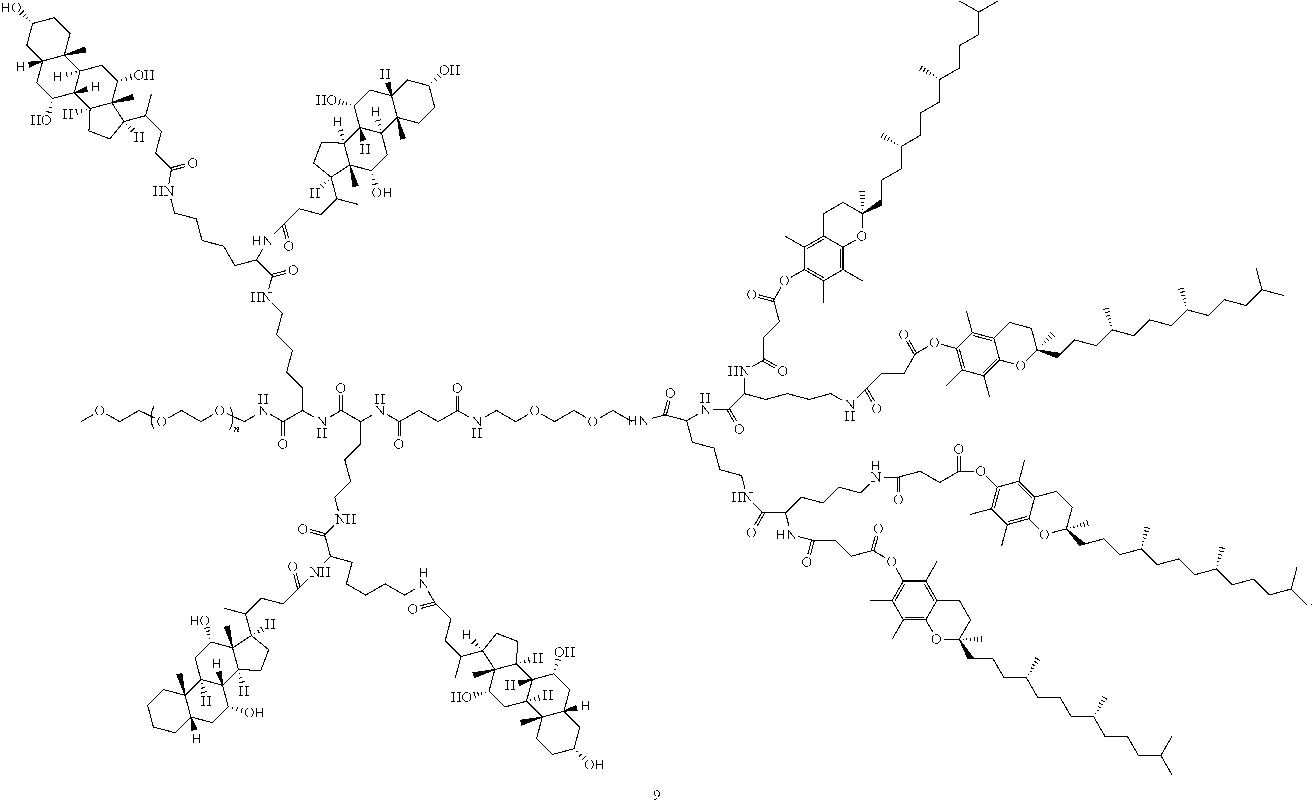

A route of telodendrimer PEG^{nk}CA₄-L-VE₄ synthesis is shown above in scheme 3.

Preparation of CMT-3 loaded Micelles: Hydrophobic CMT-3 molecules were encapsulated into the core of micelles by thin film hydration method. Both CMT-3 and VE or URA-containing telodendremers at 1:10 or 1:20 mass ratio was dissolved in DCM and methanol (MeOH) (10:1 v/v) in a 10 mL round-bottom flask. A thin film was prepared by organic solvent roto-evaporation under vacuum and further dried via oil vacuum pump. PBS buffer was then added to hydrate thin film followed by vortex and sonication. The particle size of CMT-3 encapsulated micelles was evaluated by DLS. As shown in Table 1 below, CMT-3 was successfully encapsulated by PEG^{5k}CA₄VE₄, PEG^{5k}CA₄URA₄, PEG^{5k}CA₄-L-VE₄, and PEG^{20K}CA₄-L-VE₄ nanocarriers at 0.5:10 and 1:10 mass ratio with homogeneous particle size. The particle sizes of these nanoformulations at lower CMT-3 concentrations were significantly smaller than their particle sizes at higher CMT-3 concentration.

TABLE 1
Particle size of CMT-3 nanoformulations

| | Size | | Blank |
|---|---|---|---|
| | (0.5:10 w/w) | Size | polymer |
| Formulation | (nm) | (1:10 w/w)*** | (nm) |
| CMT-3-PEG^{5k}CA₄VE₄ | 20 ± 1* | 478 ± 15 | 30 ± 1 |
| CMT-3-PEG^{5k}CA₄URA₄ | 18 ± 1* | 1469 ± 126 | 15 ± 1 |
| CMT-3-PEG^{20k}CA₄VE₄ | 62.18% 28 nm | 1143 ± 51 | 30 ± 2 |
| | 37.37% 920 nm* | | |
| CMT-3-PEG^{5k}CA₄-L-VE₄ | 54 ± 2** | 60 ± 2 | 48 ± 2 |
| CMT-3-PEG^{20k}CA₄-L-VE₄ | 40 ± 7** | ND | 38 ± 6 |

*CMT-3 concentration is 0.25 mg/mL,
**CMT-3 concentration is 0.5 mg/mL,
***CMT-3 concentration is 1 mg/mL

In vitro CMT-3 Release. CMT-3 encapsulated nanoformulations was prepared to evaluate in vitro drug release profile. 330 μL CMT-3 nanoformulation was aliquoted into each dialysis cartridges with 3.5 kDa MWCO. The cartridges were dialyzed against 50 mL of PBS buffer at 37° C., and PBS buffer was refreshed every 4 h during first 10 h and then every 8 h. The drug remained in the dialysis cartridge was detected at different time points by UV-vis spectrophotometry. The release profile was presented as mean of triplicate values with standard deviations. Referring now to FIG. 15, a drug release profile of CMT-3-PEG^{5k}CA₄VE₄, CMT-3-PEG^{20k}CA₄VE₄, CMT-3-PEG^{5k}CA₄-L-VE₄ in PBS at 37° C. is shown. In embodiments, nanocarrier sustained CMT-3 release for 24 h. Nearly 50% of CMT-3 released out at first four hours, the rest of the drug release slower during 4-24 h, indicating lower systemic toxicity of nanoformulation.

### Example II

Methods: C57BL/6 mice received aerosolized intratracheal nCMT-3 or saline, then intratracheal LPS or saline 2 h later. Tissues were harvested at 24 h. The effects of LPS and nCMT-3 on ALI were assessed by lung histology, MMP level/activity (zymography), NLRP3 protein and activated caspase-1 levels. Blood and bronchoalveolar lavage fluid (BALF) cell counts, PMN elastase, and soluble triggering receptor expressed on myelocytes-1 (sTREM-1) levels, TNF, IL-1, IL-6, IL-18 and BALF protein levels were also measured. Results: LPS-induce ALI was characterized by histologic lung injury (PMN infiltration, alveolar thickening, edema and consolidation) elevated proMMP-2, -9 levels and activity, NLRP-3 protein and activated caspase-1 levels in lung tissue. LPS-induced increases in plasma and BALF levels of sTREM-1, TNF-α, IL-1β, IL-6, IL-18, PMN elastase and BALF protein levels demonstrate significant lung/systemic inflammation and capillary leak. Intratracheal nCMT-3 significantly ameliorated all of these LPS-induced lung injury/inflammation markers, to control levels in most instances. Conclusions: Collectively the results suggest pretreatment with nCMT3 significantly attenuates LPS-induced lung injury/inflammation by multiple mechanisms including: MMP activation, PMN elastase, sTREM-1 release and NLRP3 inflammasome/caspase-1 activation. Key Words: LPS, ARDS, tetracycline, inflammasome, MMP, sTREM-1

Acute respiratory distress syndrome is a life-threatening complication of sepsis with significant morbidity and mortality. In ARDS, an exaggerated inflammatory response to infection disrupts pulmonary capillaries and air space integrity resulting in alveolar flooding and pulmonary edema. The heterogeneous lung injury in ARDS results in stiff, noncompliant lungs which are difficult to ventilate and oxygenate. Current treatment of sepsis-related ARDS incudes antibiotics to fight infection, fluid resuscitation and vasopressors to maintain blood pressure, and interventions like mechanical ventilation and dialysis to support lung and kidney function. Despite these treatments, the mortality rate from ARDS remains high (30-40%), because supportive measures currently available fail to effectively address the underlying role of systemic inflammation in the pathogenesis of the disease.

Chemically modified tetracyline (6-demethyl-6-deoxy-4dedimentylamino-tetracycline:CMT-3) is a nonantibacterial, anti-inflammatory agent with pleiotropic effects including: inhibition of MMP-2 and -9, neutrophil elastase and inflammatory cytokines as well as increase in tissue inhibitor of metalloproteinase (TIMP)-1⁴. CMT-3 has been shown to attenuate sepsis-induced inflammation and lung injury in cecal-ligation puncture (CLP) and porcine models of ARDs. Unfortunately, CMTs are only available for oral administration and their hydrophobicity, poor solubility and limited absorption through the intestinal tract limit their efficacy in ALI/ARDS. Nanomaterial-based delivery systems have been broadly applied in preclinical studies and clinical applications to increase bioavailability, reduce toxicity and improve pharmacokinetics via enhanced delivery, which are promising to increase the bioavailability and pharmacological activity of anti-inflammatory compounds. However, none perform in accordance with those described in accordance with the present disclosure.

The lung is an attractive route for non-invasive drug delivery, either locally or systematically, with many advantages, such as a high surface area with rapid absorption due to high vascularization and circumvention of the first pass effect. Incorporating polymer nanoparticles to therapeutic drugs provides an additional degree of manipulation for drug delivery systems, while also enabling sustained release and targeting of specific cells and organs. Telodendrimer is a linear-dendritic block copolymer with a well-defined dendritic domain for customized nanocarrier design for different therapeutic molecules by introducing various drug-binding molecules on the dendritic periphery. Aerosolization or inhalation of drug delivery systems from lung are currently being extensively studied and have great potential for targeted drug delivery in the treatment of a variety of diseases. With that in mind a nano-formulation of CMT-3 (nCMT-3) of the present disclosure was developed using telodendrimers to be given via aerosol administration as an anti-inflammatory agent to reduce systemic inflammation and lung injury in ARDS.

To test the nCMT-3 preparation of the present disclosure its ability to prophylactically attenuate lung injury and systemic inflammation was examined in a murine model of ARDS caused by intratracheal LPS administration. The results evidence nCMT-3 is effective in attenuating histologic lung injury and capillary leak (BALF protein levels). It is also shown that nCMT-3 attenuates MMP levels and activation, triggering receptor expressed on myeloid cells-1 (TREM-1) and expression of the NLRP3 inflammasome/activated caspase-1 in lung tissue. TREM-1 is expressed on neutrophils and monocyte/macrophages and amplifies Toll-like receptor (TLR)-mediated inflammation during infection. The NLRP3 inflammasome activates proinflammatory cytokines (IL-1β and IL-18) and caspase-1 mediated lung injury. Collectively the data provides evidence prophylactic nCMT-3 attenuates LPS-mediated lung injury and inflammation via multiple anti-inflammatory mechanisms including: MMP activation, PMN elastase, sTREM-1 release and NLRP3 inflammasome/caspase-1 activation.

Materials and Methods: CMT-3 was obtained from CMTx Biotech Inc. Methoxypolyethylene glycol amin 5000 (MeO-PEG^{5k}-NH₂) was purchased from Jenkem. (Fmoc)-Lys(Fmoc)-OH, (Fmoc)-Lys¹³-OH, and N-hydroxybenzotriazole (HOBt) were purchased from AnaSpec Inc. Cholic acid (CA), D-α-tocopherol succinate, diisopropyl carbodiimide (DIC), N,N-Dimethylformamide (DMF), dichloromethane (DCM), trifluoroacetic acid (TFA) and other chemical reagents were acquired from Sigma-Aldrich. Dialysis membrane tubing 3500 Dalton molecular weight cut-off (MWCO) was bought from Spectrum Laboratories Inc. MALDI-TOF MS and H NMR of PEG^{5k}CA₄VE₄ Telodendrimer. Mass spectra of PEG^{5k}CA₄VE₄ was obtained by Bruker Microflex MALDI-TOF. ¹H NMR spectra was collected from Bruker600 MHz nuclear magnetic resonance using DMSO-d₆ as solvent. The characterization was demonstrated in a previous paper. Particle size distribution of CMT-3-PEG^{5k}CA₄VE₄ were detected by Zetatrac dynamic light scattering (DLS) instrument (Microtrac Inc.) with 20 mg/mL telodendrimer concentration at room temperature. The data was analyzed by volume distribution via Microtrac FLEX Software version 10.6.0. Transmission electron microscopy (TEM) images were taken on a JEOL JEM-1400 instrument at 80 kV. The samples solution was dropped onto carbon coated grids and negatively stained by uranyl acetate. Separation and detection were carried out by HPLC system (Shimadzu, Japan) equipped with UV detector. 10 μL sample was injected into the HPLC system. A C18 column (Shimadzu, Japan) was utilized with water and acetonitrile as mobile phase A and B, respectively under a gradient program at 1 mL/min flow rate with 458 nm detection.

Telodendrimer PEG^{5k}CA₄VE₄ Preparation: As shown in Scheme 1 (route of teleodendrimer synthesis) (See also FIG. 2), telodendrimer including four cholic acid and D-α-tocopherol moieties was prepared through solution phase peptide synthesis starting from MeO-PEG^{5k}-NH₂ (1). Fmoc-Lys(Fmoc)-OH (three equiv) was conjugated onto NH₂ group of PEG^{5k} via coupling reagents HOBt and DIC confirming by negative Kaiser test result. PEGylated materials (2) were precipitated by pouring chilled ethyl ether into reaction solution and washed by chilled ethyl ether twice. Fmoc group was de-protected by 20% (v/v) 4-methylpiperidine in DMF, and the product was precipitated and washed three times by chilled ethyl ether. After vacuum drying at room temperature, one (Fmoc)-Lys(Fmoc)-OH (3) and (Fmoc)-Lys¹³-OH (4) were coupled sequentially to achieve dendritic polylinsine intermediate with four Fmoc and Boc moieties. To obtain PEG^{5k}CA₄VE₄, four cholic acid groups were conjugated on α-NH₂ of lysine (5) after Fmoc groups removal, while D-α-tocopherol succinate moieties were coupled on the ε-NH₂ (6) after Boc de-protection via 50% TFA (v/v) in DCM.

Preparation of CMT-3 loaded Micelles: Hydrophobic CMT-3 molecules were encapsulated into the core of micelles by thin film hydration method. Both CMT-3 and PEG5kCA4VE4 at 1:20 mass ratio was dissolved in DCM and methanol (MeOH) (10:1 v/v) in a 10 mL round-bottom flask. A thin film was prepared by organic solvent roto-evaporation under vacuum and further dried via oil vacuum pump. 1 mL PBS buffer was then added to hydrate thin film followed by vortex and sonication. The particle size of CMT-3 encapsulated micelles was evaluated by DLS.

In vitro CMT-3 Release: CMT-3 encapsulated PEG^{5k}CA₄VE₄ was prepared to evaluate in vitro drug release profile. 330 μL CMT-3-PEG^{5k}CA₄VE₄ nanoformulation was aliquoted into each dialysis cartridges with 3.5 kDa MWCO. The cartridges were dialyzed against 50 mL of PBS buffer at 37° C., and PBS buffer was refreshed every 4 h during first 10 h and then every 8 h. The drug remained in the dialysis cartridge was detected at different time points by UV-vis spectrophotometry. The release profile was presented as mean of triplicate values with standard deviations.

Cell Viability Assay: Macrophage cell RAW 264.7, kidney epithelial cell 293T and Monocyte THP-1 were purchased from American Type Culture Collection (ATCC, USA) and were culture in DMEM or RPMI-1640 with 10% fetal bovine serum (FBS), 100 μg/mL streptomycin and 100 U/mL penicillin G at 5% CO₂, 37° C. in a humidified incubator. Cells were seeded in 96-well plates at a density of 6000 (RAW 264.7 and THP-1) and 4000 (293T) cells/well. After overnight incubation, free CMT-3, CMT-3-PEG^{5k}CA₄VE₄, and blank PEG^{5k}CA₄VE₄ with different concentrations were added into each well and incubated for 48 and 72 h, respectively. CellTiter 96© Aqueous Cell Proliferation Reagent composed of MTS and PMS reagents was added into each well according to the manufacturer instruction and further incubated at 37° C. for 2 h in RAW 264.7 and 293T cells and 4 h in THP-1 cells. The cell viability was evaluated by UV-absorbance at 490 nm in a microplate reader (BioTek Synergy H1). The untreated cells are considered as control. Results were calculated from the equation cell viability %=(ODtreat−ODblank)/(ODcontrol−ODblank)×100% (n=3).

Pharmacokinetic and Biodistribution Study: Healthy C57BL/6J mice aged 4-5 weeks were administrated intratracheally (n=3) or intravenously (n=2) with CMT-3-PEG^{5k}CA₄VE₄ nanoformulation at a CMT-3 single dose of 1 mg/kg (I.T.) or 2 mg/kg (I.V.) body weight, respectively. Mice were euthanized at 10 min, 15 min, 30 min, 2 h, 4 h, 8 h, and 24 h. Blood and the organs including heart, kidney, liver, lung, and spleen were collected from mice at different time points. Plasma was collected in a heparinized tube and isolated by centrifugation. The organs were washed by PBS and homogenized with extraction buffer (10% Triton X-100, deionized water, and acidified isopropanol (0.75 N HCl) at 1:2:15 v/v/v) by tissue grinder.

Standard samples of plasma and tissues: Plasma: Plasma specimen was collected from Balb/c mice obtained and stored at −80° C. To construct a calibration curve, different amount CMT-3 were added to 50 μl of blank plasma with 150 μl PBS to obtain CMT-3 standard concentrations ranging from 0.5 to 50 μg/ml in plasma.

Tissue homogenates: 50 mg heart, kidney, liver, lung, and spleen tissues of Balb/c mice were homogenized with 2-fold weight of PBS and 5-fold weight of extraction buffer. 350 μl aliquots of homogenates were spiked with CMT-3 solutions to prepare homogenates with CMT-3 concentrations ranging from 0.5 to 50 μg/ml.

Solid phase extraction: 200 μL Plasma sample was acidified using 8 μl of 50% phosphoric acid and vortex-mixed for 30 s. Tissue homogenates: heart, kidney, liver, lung, and spleen tissues of Balb/c mice were homogenized with 7-fold weight of extraction buffer and incubated at −20° C. overnight. The homogenate samples or plasma samples were spined down and the supernatant was transferred onto C18 extraction cartridge (Waters Corporation, Milford, MA, USA) that was conditioned and equilibrated by washing with 1 ml of methanol (100, v/v) and 1 ml of Milli-Q water. The cartridge was washed with 0.5 ml of methanol/water (5/95, v/v). Analytes were eluted with 2 ml of methanol (100, v/v). After evaporation, the samples were reconstituted with 125 μL acetonitrile. 70 μL acetonitrile solution was taken and was added 30 μL water. The resulting solution was injected into the high-performance liquid chromatography (HPLC) system.

Animals and lung injury model: Male and female C57BL/6 mice (age: 8 weeks) were purchased from Jackson Laboratories (Bar Harbor, ME). All animals were housed under controlled temperature (22°) and photoperiod (12-h light and 12-h dark cycle) with free access to water and food. The animal experiments were approved by the Institutional Animal Care and Use Committee of the SUNY Upstate Medical University (IACUC #344). The study was performed in accordance with the National Institutes of Health and ARRIVE guidelines on the use of laboratory animals.

Acute lung injury induction, nCMT-3 treatment and tissue harvest: All mice underwent non-invasive tracheal installation by aerosolizer with nCMT-3 (1 mg/kg) or vehicle 2 h before induction of lung injury by LPS or saline as described previously¹⁵. Briefly, the mice in the septic and control groups were anesthetized by intraperitoneal injection with a combination of ketamine (80 mg/kg) and xylazine (8 mg/kg). Once adequate anesthesia is observed, mice were suspended by their incisors in the supine position on the intubating platform and the fiber-optic illuminator was turned on and positioned over the trachea. Curved blunt-ended forceps were used to carefully grasp the tongue and in an upward and leftward motion, the tongue was positioned to gain visualization of the larynx. Hands-free binocular magnifiers can be used for improved visualization of the larynx. MicroSprayer Aerosolizer (Cat. #: YAN30012, Shanghai Yuyan Instruments Co., Ltd) was inserted the trachea and LPS, nCMT-3 and saline solution (volume not to exceed 70 μl per mouse) were instilled. The mouse was maintained in the same position on the intubating platform for at least 30 s, and then placed it prone on a heating pad for recovery. All surviving mice were sacrificed at 24 h post LPS, then blood (EDTA used as anticoagulant), lung tissue (either fixed with 10% formalin lung histology or frozen for protein analysis) and bronchoalveolar lavage fluid (BALF) were collected.

Hematological analysis: Different white blood cells were counted using a hematology analyzer (HEMAVET 950 FS). Percentages of monocyte (MO %), lymphocyte (LY %), and neutrophils (NE %) were calculated to represent changes in white blood cells. In addition, neutrophil/lymphocyte ratio (NLR %) was calculated to determine the prognosis of an inflammatory reaction by treatment.

Cytological analysis in BALF: BALF was obtained from mouse lung and lavaged with 3×0.5 ml of sterile saline, and then centrifuged at 250×g for 10 min. The pellet was resuspended with 1 ml of sterile saline. 100 μl of cell suspension were centrifuged by cytospin centrifuge (Hettich ROTOFIX 32A) at 1000 rpm for 3 min to mount the cells on a slide. The slide was air-dried and stained with Hema-3 (Fisher Scientific, Kalamazoo, MI) for analysis. Neutrophils and macrophages were counted using Nikon Eclipse TE2000-U research microscope (Nikon, Melville, NY).

Protein extraction and protein assay: Frozen lung was homogenized in RIPA buffer and extracted protein was used for Western blot analysis. Total protein concentrations from lung and BALF were determined by the BCA micro assay kit (Thermo Scientific, Rockford, IL).

Western Blot: 20 μg of protein were separated by SDS-PAGE gel, then transferred to PVDF membranes (Millipore Co., Ltd. USA). The membranes were incubated with 5% non-fat milk (Bio-Rad Laboratories) in Tris-buffered saline plus 0.5% Tween-20 (TBS-T) for 1 h at room temperature, and then overnight at 4° C. with primary antibodies purchased from Santa Cruz Biotechnology, including caspase-1 (Cat. #: sc-56036, 1:200 dilution) and NLRP3 inflammasome (Cat. #: sc-134306, 1:200 dilution). The secondary antibody linked to horseradish peroxidase (HRP) purchased from Santa Cruz Biotechnology (Cat. #: 1662408, Bio-Rad Laboratories) was applied for 1 h at room temperature. Antibody-antigen complexes were visualized using ECL according to the manufacturer's instructions. The images were analyzed quantitatively by densitometry with Image J software. The relative density of immunoreactive bands was normalized to the density of the corresponding GAPDH bands.

ELISA: Blood sample and BALF were collected for the measurements of TNF-α, (Cat. #; 50-112-8800, Invitrogen), IL-1β (Cat. #: 50-112-8814, Invitrogen), IL-6 (Cat. #: 50-112-8863, Invitrogen), TREM-1 (Cat. #: EMTREM1, Thermo Fisher Scientific Inc.), IL-18 (Cat. #: BMS618-3, Invitrogen). All cytokines were measured using commercial ELISA's kits according to the manufacturer's instructions.

Histological assessment of lung injury: Lungs were inflation-fixed by means of tracheal instillation of 0.5 ml of 10% neutral formalin. Fixed lungs were embedded in paraffin. 5 μm sections of lung tissues were stained with Hematoxylin and Eosin (H&E). Histopathology was evaluated by two independent pathologists. The histopathological assessment of acute lung injury was performed using a 0-2 scoring system described in a previous study, Briefly, neutrophils in the alveolar space and in the interstitial space were counted separately. Hyaline membranes, proteinaceous debris filling the airspaces and septal thickening were evaluated. To generate a lung injury score, the sum of each of the five independent parameters weighted according to the relevance ascribed to each feature and then were normalized to the number of fields evaluated. Three fields per slide were counted at ×400 magnification under light microscopy.

Gelatin zymography: Gelatin zymography was used to examine activity of MMP-2 and MMP-9 in lung tissues. 10 μg of total protein were subject to electrophoresis on sodium dodecyl sulfate-polyacrylamide gel electrophoresis gels that contained 0.1% gelatin as a substrate for MMP digestion. After electrophoresis, the gels were incubated 24 h, then stained with Coomassie Blue, and MMP activity was evident as cleared bands of substrate lysis. The MMPs were identified by their molecular weights and inhibition by ethylenediaminetetraacetic acid or phenanthroline. Activity was quantitated by scanning densitometric analysis using with NIH Image J software

Statistical analysis: The data are expressed as mean±SEM. The data analysis was performed using GraphPad Prism software (version 5.0). The sample size for each experimental group (n=x) is presented in the figure legend. One-way analysis of variance (one-way ANOVA) with Bonferroni's multiple comparisons test was used to determine group differences. Differences among groups were considered significant at P<0.05. All data was obtained from three or more independent experiments.

RESULTS: Following the publication, a hybrid telodendrimer with both cholic acid (CA) and vitamin E(VE) as peripheral groups (Scheme 1) was synthesized to increase drug binding as well as increase micelle stability by the amphiphilic CA. As shown in FIG. 5A, CMT-3 was successfully encapsulated by PEG^{5k}CA₄VE₄ nanocarrier at 1:20 mass ratio with homogeneous particle size at 27 nm. The small particle size of the nanoformulation allows convenient administration of CMT-3-PEG^{5k}CA₄VE₄ through i.v. or i.t. injection. TEM images shown in FIG. 5B reveal the spherical particle morphology of blank PEG^{5k}CA₄VE₄ and CMT-3-PEG^{5k}CA₄VE₄. The drug release profile relates the stability of the drug encapsulated nanoformulations. Since CMT-3 is barely dissolved in aqueous solutions, the drug release profile of CMT-3-PEG^{5k}CA₄VE₄ nanoformulation was evaluated. In FIG. 5C, nanocarrier sustained CMT-3 release for 24 h. Nearly 50% of CMT-3 released out at first four hours, the rest of the drug release slower during 4-24 h, indicating lower systemic toxicity of nanoformulation. The cytotoxicity assay on various normal cells shown in FIG. 5D indicate that blank PEG^{5k}CA₄VE₄ telodendrimer is nontoxic up to 1 mg/mL. In comparison with free CMT-3, CMT-3-PEG^{5k}CA₄VE₄ nanoformulation significantly reduce the drug toxicity in 293T cells at 10 μg/mL concentration, demonstrating lower kidney toxicity of nanoformulation. In addition, CMT-3-PEG^{5k}CA₄VE₄ slightly decrease drug cytotoxicity in RAW 264.7 and THP-1 cells, which may also facilitate their retention in the lung through i.t. injection.

More Specifically, FIGS. 5A-5D depicts in vitro characterization of CMT-3-PEG^{5k}CA₄VE₄ nanoformulation. FIG. 5A depicts particle size of CMT-3-PEG^{5k}CA₄VE₄ (nCMT-3) measured by DLS particle sizer. FIG. 5B depicts particle morphology of empty PEG^{5k}CA₄VE₄ nanoparticle and CMT-3-PEG^{5k}CA₄VE₄ nanoformulation detected by TEM after negative staining by uranium acetate. The scale bar of TEM image is 50 nm. FIG. 5C depicts drug release profile of CMT-3-PEG^{5k}CA₄VE₄ in PBS at 37° C. FIG. 5D depicts cell viability assay of CMT-3, CMT-3-PEG^{5k}CA₄VE₄ and PEG^{5k}CA₄VE₄ in RAW 264.7, THP-1 and 293T cell lines.

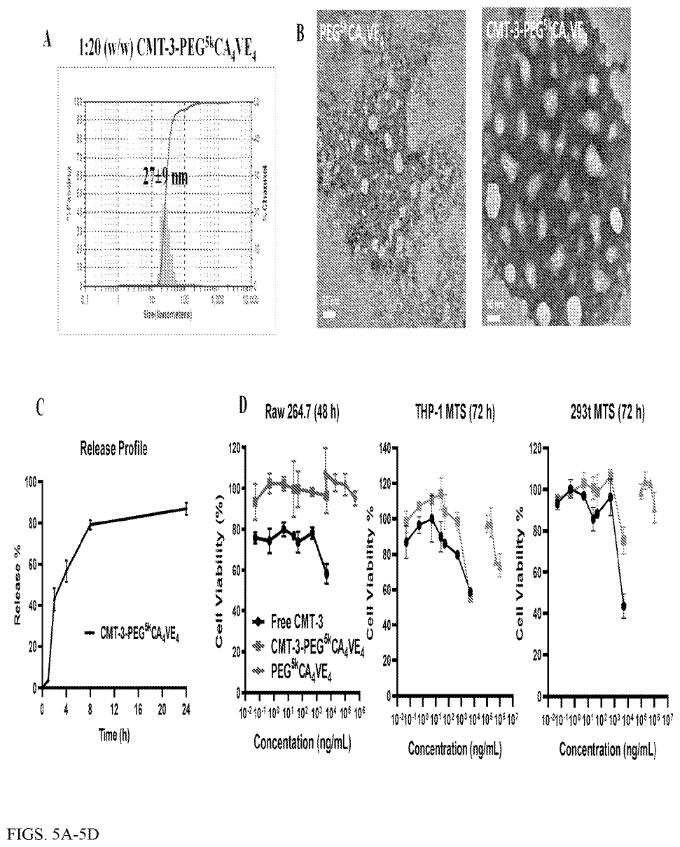

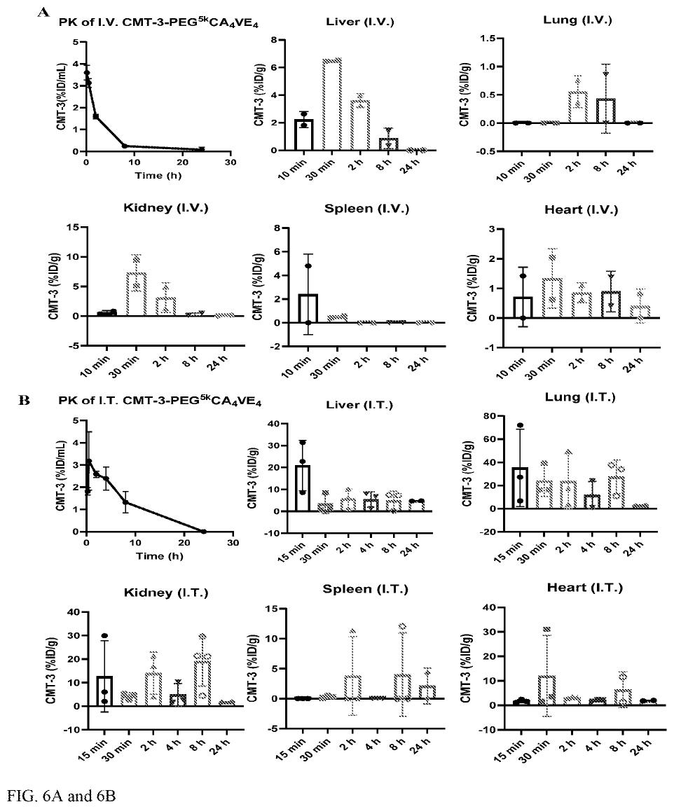

Pharmacokinetics and biodistribution of nano-CMT-3 formulations: The pharmacokinetic profiles and biodistribution of CMT-3-PEG^{5k}CA₄VE₄ nanoformulation were investigated in C57BL/6J mice after intravenous (i.v.) or intratracheal (i.t.) administration. As shown in FIG. 6A, plasma drug concentration decay right after iv injection the less amount of drug in the plasma as expected; while plasma concentration of drug peaked at 30 min post i.t. injection and started to decay in a relatively slow rate compared to iv injection, which indicating that drug molecules continuously diffuse into systemic from lung in i.t. injection (FIG. 6B). The obvious difference in CMT-3 concentration in lung was observed between i.t. and i.v. injection. After i.t. injection, nanoformulation reside in lung about eight hours at a significantly high concentration about 20% injected dose; while dug in lung were only detectable at 2 h and 8 h post i.v. injection at <1% injection dose level. The high drug concentration in lung after i.t. injection may project for effective anti-inflammatory effect in protecting lung tissue in ARDS. Interestingly, the drug concentration in other organs after i.t. injection, e.g. liver, kidney, heart and spleen, were also significantly higher than i.v. injection, due to the slow drug diffusion/release from lung as a drug depot, which also prevents remote organ inflammation and damage in sepsis.

More specifically, FIGS. 6A and 6B depict in vivo pharmacokinetics and biodistribution of CMT-3-PEG^{5k}CA₄VE₄ nanoformulation after (FIG. 6A) intravenous injection (IV) or (FIG. 6B) intratracheal injection (IT): CMT-3 concentration in plasma and major organs (liver, lungs, kidney, spleen and heart) was analyzed by HPLC after solid-phase extraction.

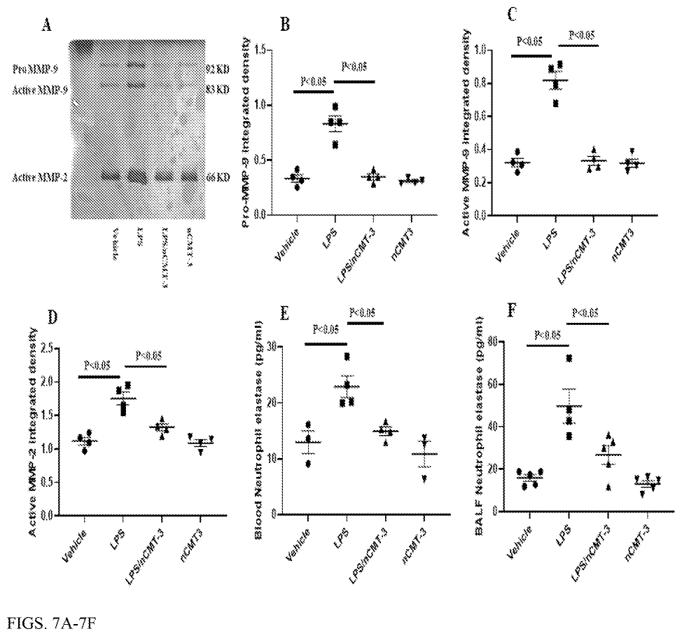

nCMT-3 inhibited MMP activities and the levels of neutrophil elastase in LPS-induced ALI: CMT-3 as an MMP inhibitor can reduce MMP-2 & 9, neutrophil elastase activity in lung injury. First, nCMT-3 was administered by intratracheal injection to determine if nCMT-3 could block MMPs and neutrophil elastase activity in ALI by LPS. MMP 2 & 9 by gelatin zymography were detected (FIG. 7A), showing that treatment with nCMT-3 significantly reduced pro-MMP-9 (FIG. 7B), active MMP-9 (FIG. 7C), and active MMP-2 (FIG. 7D) (LPS vs. LPS/nCMT-3, P<0.05). Increased neutrophil elastase activity by LPS in plasma (FIG. 7E) and BALF (FIG. 7F) were decreased by nCMT-3 significantly (LPS vs. LPS/nCMT-3, P<0.05). These results confirm that intratracheal administration of nCMT-3 has similar functionality to free CMT-3 in inhibiting MMPs and neutrophil elastase activity.

More specifically, FIGS. 7A-7F depict Matrix metallopeptidase 2 & 9 (MMP-2 & 9) levels lung tissue and the levels of neutrophil elastase (NE) in blood and BALF. Mice were treated with Nano-CMT-3 (1 mg/kg) or vehicle 2 h before induction of lung injury by LPS (2.5 mg/kg) or sham lung injury (by saline) using non-invasive tracheal installation by aerosolizer. Mice were sacrificed 24 h after LPS or saline, then lung tissue was collected for protein isolation. Gelatin zymography was used to detect MMP-2 and MPP-9. Plasma and BALF were collected for NE by ELISA. Representative gelatin zymography showed the differences in pro MMP-9/active MMP-9 and active MMP-2 in panel A. The abundances of pro MMP-9 (FIG. 7B), active MMP-9 (FIG. 7C) and active MMP-2 (FIG. 7D) were quantified using Image J. The levels of NE in plasma (FIG. 7E) and BALF (FIG. 7F) were assayed. Scatter dot plot represents mean values and standard error of mean (SE) (n=4/group).

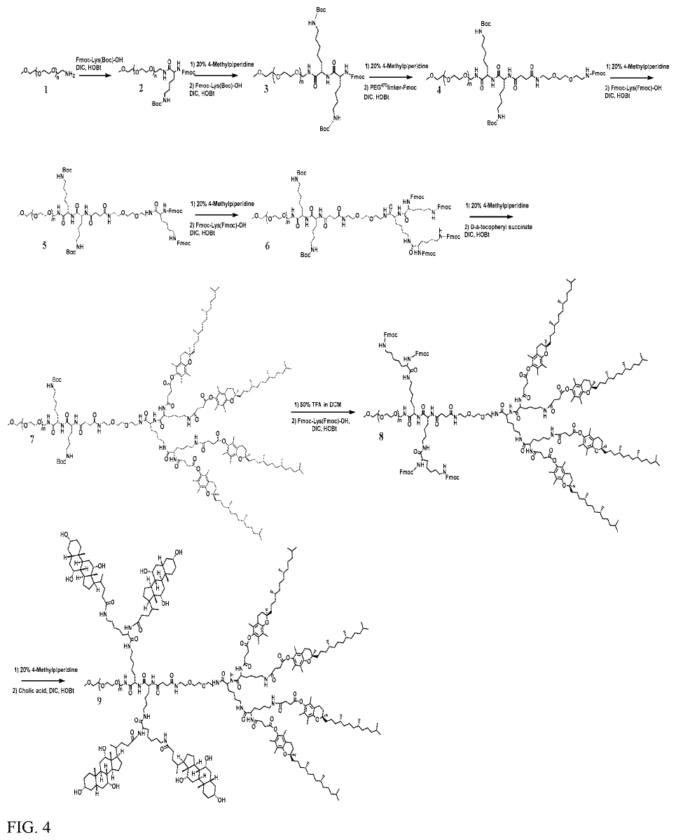

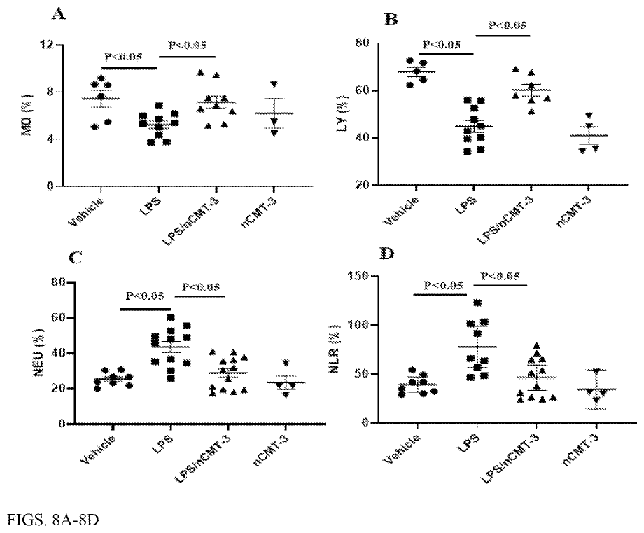

nCMT-3 regulates inflammation, impacts BALF neutrophil and macrophage and improves lung injury in LPS-induced ALI: Cytokines are the main cellular component of the inflammatory and immune response that protects against infection. Cytokines produced by several immune cells are regulators of host responses to infection, immunity and inflammation. The release of proinflammatory cytokines activate immune cells, leading to the further release of cytokines. The effects of nCMT-3 administration on leukocytes (monocyte, lymphocyte and neutrophil) and cytokine levels (TNF-α, IL-1β, IL-6 and IL-18) in BALF and plasma was examined to assess its effects on systemic inflammation after ALI. Decreased percentage of monocytes (MO %, FIG. 8A) and percentage of lymphocytes (LY %, FIG. 8B) observed in LPS mice were attenuated by nCMT-3 administration. Increased percentage of neutrophils (NEU %, FIG. 8C) and neutrophil-to-lymphocyte ratio (NLR %, FIG. 4D) were restored by nCMT-3.

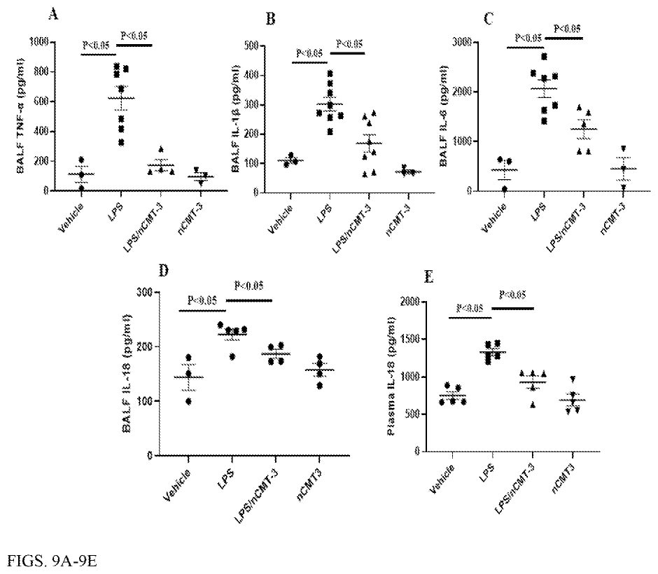

More specifically, FIGS. 8A-8D depict a hematological analysis. Here, mice were treated with Nano-CMT-3 (1 mg/kg) or vehicle 2 h before induction of lung injury by LPS (2.5 mg/kg) or sham lung injury (by saline) using non-invasive tracheal installation by aerosolizer. Mice were sacrificed 24 h after LPS or saline, then blood was collected for blood cell counting. Percentage of monocytes (MO %) (FIG. 8A), percentage of lymphocytes (LY %) (FIG. 8B), percentage of polymorphonuclear leukocytes (PMNs %) (FIG. 8C) and neutrophil-to-lymphocyte ratio (NLR %) (FIG. 8D) were calculated. Scatter dot plot represents mean values and standard error of mean (SE) (n=3-12/group). nCMT-3 significantly attenuated the increase in TNF-α (FIG. 9A), IL-1β (FIG. 9B), IL-6 (FIG. 9C) and IL-18 (FIG. 9D) in BALF and IL-18 (FIG. 9E) in plasma (LPS vs. LPS/nCMT-3, P<0.05).

More specifically, FIGS. 9A-9E depict cytokine levels in bronchoalveolar lavage fluid (BALF) and plasma. Here, mice were treated with Nano-CMT-3 (1 mg/kg) or vehicle 2 h before induction of lung injury by LPS (2.5 mg/kg) or sham lung injury (by saline) using non-invasive tracheal installation by aerosolizer. Mice were sacrificed 24 h after LPS or saline, then BALF and plasma were collected for cytokines by ELISA. TNF-α (FIG. 9A), IL-1β (FIG. 9B), IL-6 (FIG. 9C) and IL-18 (FIG. 9D) in BALF and IL-18 (FIG. 9E) in plasma were assayed. Scatter dot plot represents mean values and standard error of mean (SE) (n=3-12/group).

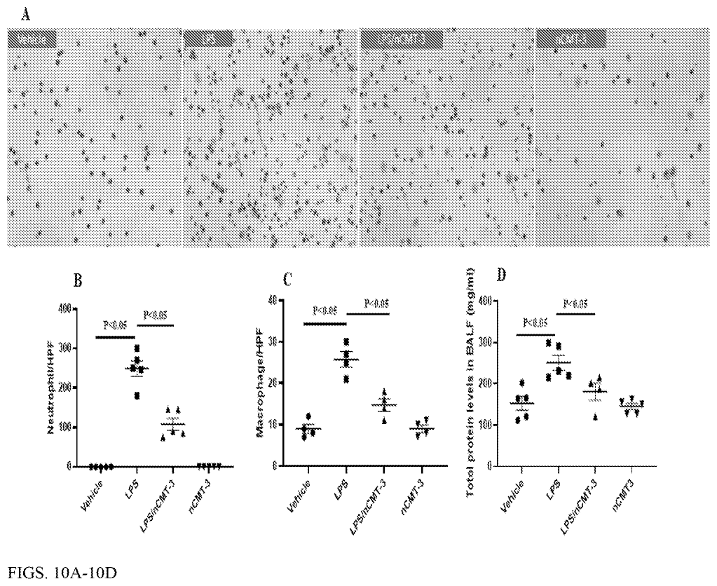

The accumulation of neutrophils in the lungs is one of the hallmarks of acute inflammatory response. Alveolar macrophages can act as phagocytes and release a wide variety of biologically active molecules in ALI. Neutrophils and macrophages were observed by hema-3 staining (FIG. 10A) in BALF. Neutrophils (FIG. 10B) and macrophages (FIG. 10C) were counted. Significant increases in neutrophils and macrophages by LPS were reduced by nCMT-3 administration (LPS vs. LPS/nCMT-3, P<0.05). Additionally, total amount of protein as shown in FIG. 10D, was lower in the LPS plus nCMT-3 group than in the LPS alone group (P<0.05).

More specifically, FIGS. 10A-D depict cytological analysis and total protein concentration in bronchoalveolar lavage fluid (BALF). Here. mice were treated with Nano-CMT-3 (1 mg/kg) or vehicle 2 h before induction of lung injury by LPS (2.5 mg/kg) or sham lung injury (by saline) using non-invasive tracheal installation by aerosolizer. Mice were sacrificed 24 h after LPS or saline, then BALF was collected for examining neutrophil and macrophage by Hema 3 staining and total protein concentration by BCA. Representative Hema3-stained image for neutrophil (orange arrows) and macrophage (green arrows) was shown in panel A (FIG. 10A). Quantification of neutrophils (FIG. 10B) and macrophages (FIG. 10C) per slide were counted at ×400 magnification under light microscopy. Total amount of protein (FIG. 10D) was assayed. Scatter dot plot represents mean values and standard error of mean (SE) (n=4-5/group).

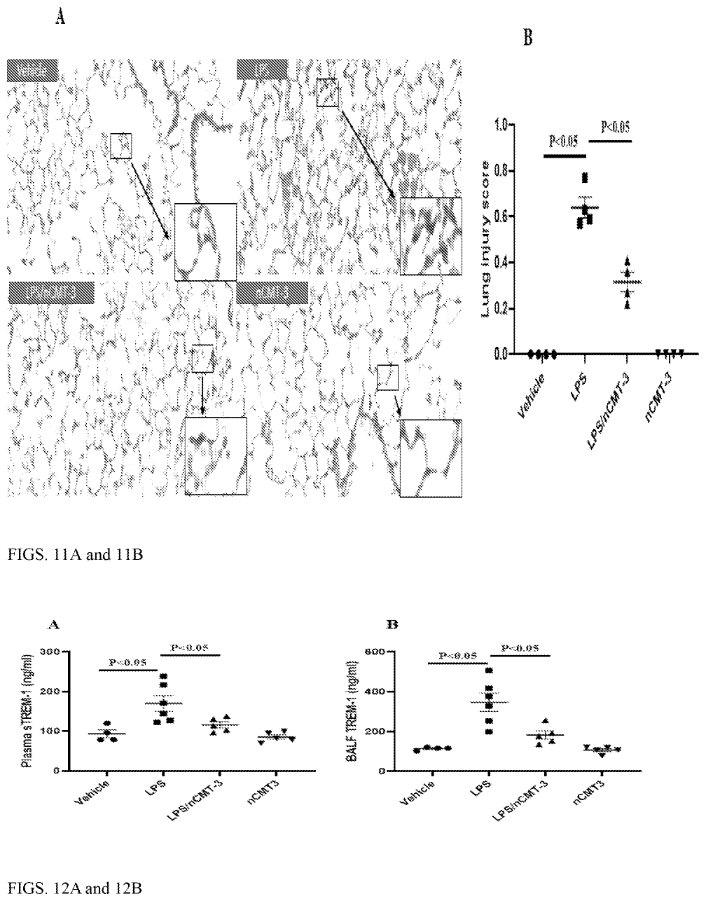

Lung histological assessment was used to evaluate lung injury as shown in FIG. 11A. Standard assessment method was used as described in the section of methods. Lung injury score was calculated (FIG. 11B) and showed that lung injury score was significantly attenuated by nCMT-3 (LPS vs. LPS/nCMT-3, P<0.05).

More specifically, FIGS. 11A and 11B depict a histological assessment of lung injury. Here, mice were treated with Nano-CMT-3 (1 mg/kg) or vehicle 2 h before induction of lung injury by LPS (2.5 mg/kg) or sham lung injury (by saline) using non-invasive tracheal installation by aerosolizer. Mice were sacrificed 24 h after LPS or saline, then lung tissue was collected for H&E staining to evaluate lung injury from each group as shown in panel A (FIG. 11A). Lung injury were characterized by neutrophil infiltration (blue arrows), hyaline membranes, proteinaceous debris filling the airspaces and alveolar septal thickening (green arrow). Semi-quantitative histological lung injury score was assessed as shown in panel B (FIG. 11B). Scatter dot plot represents mean values and standard error of mean (SE) (n=4-5/group).

These results provide evidence that nCMT-3 administer by intratracheal injection has the same functionality as free CMT-3 in modulating systemic inflammation and improving lung injury in ALI. At the same time, these findings further confirm the effect of CMT-3 on inhibiting neutrophil infiltration/accumulation and macrophage activation in ALI.

nCMT-3 regulates sTREM-1 and NLRP3 inflammasome/caspase-1 pathways in LPS-induced ALI: TREM-1, an important signaling receptor expressed on neutrophils and monocytes, plays an important role in systemic infection. NLRP3 inflammasome mediates the activation of caspase-1 and the secretion of the proinflammatory cytokines (e.g. IL-1β and IL-18) in response to microbial infection and cellular damage. To assess potential mechanisms for modulation of lung injury by nCMT-3/free CMT-3 we measured sTREM-1, NLRP3 inflammasome and caspase-1. sTREM-1 levels were increased in plasma (FIG. 12A) and BALF (FIG. 12B) by LPS (P<0.05 vs. Vehicle) and restored in the LPS/nCMT-3 group (P<0.05 vs. LPS).

More specifically, FIGS. 12A and 12B depict levels of soluble triggering receptor expressed on myeloid cells 1 (sTREM-1) in blood and BALF. Here, mice were treated with Nano-CMT-3 (1 mg/kg) or vehicle 2 h before induction of lung injury by LPS (2.5 mg/kg) or sham lung injury (by saline) using non-invasive tracheal installation by aerosolizer. Mice were sacrificed 24 h after LPS or saline, then plasma and BALF were collected for NE and sTREM-1 by ELISA. Plasma sTREM-1 (FIG. 12A) and BALF sTREM-1 (FIG. 12B) were assayed. Scatter dot plot represents mean values and standard error of mean (SE) (n=3-5/group).

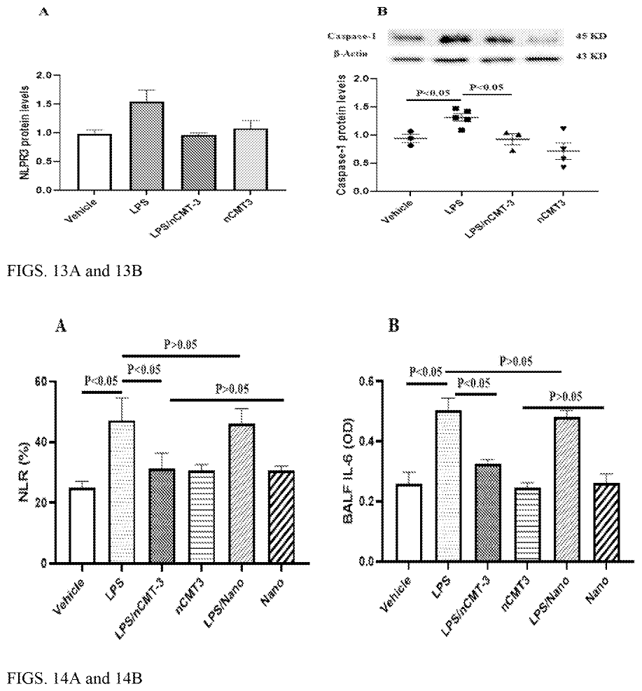

Next, levels of NLRP3 inflammasome and caspase-1 lung tissue were examined by Western blot as shown in FIGS. 13A and 13B. The elevations in NLRP3 inflammasome (FIG. 13A) and caspase-1 (FIG. 13B) observed in LPS mice were ameliorated by nCMT-3 administration. These findings suggest CMT-3 can modulate lung injury by several potential mechanisms including regulating the TREM-1 and NLRP3 inflammasome/caspase-1 axis.

More specifically, FIGS. 13A and 13B depict the levels of caspase-1 and NPRL3 inflammasome in lung tissue. Here, mice were treated with Nano-CMT-3 (1 mg/kg) or vehicle 2 h before induction of lung injury by LPS (2.5 mg/kg) or sham lung injury (by saline) using non-invasive tracheal installation by aerosolizer. Mice were sacrificed 24 h after LPS or saline, then lung tissues were harvested for protein analysis. Caspase-1 (FIG. 13A) and NPRL3 (FIG. 13B) inflammasome were measured by Western Blot. Scatter dot plot represents mean values and standard error of mean (SE) (n=3-5/group).

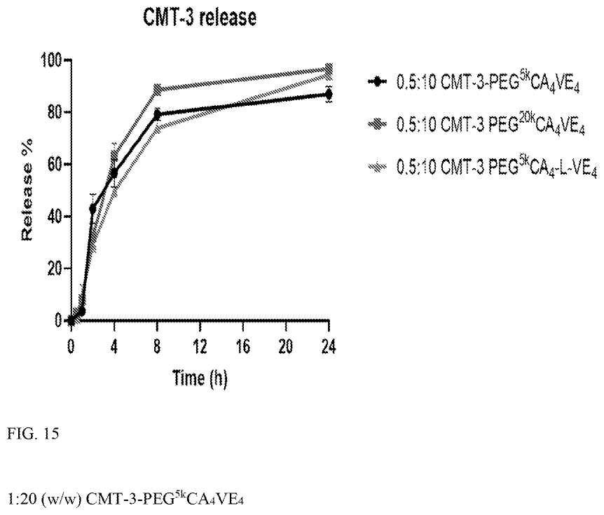

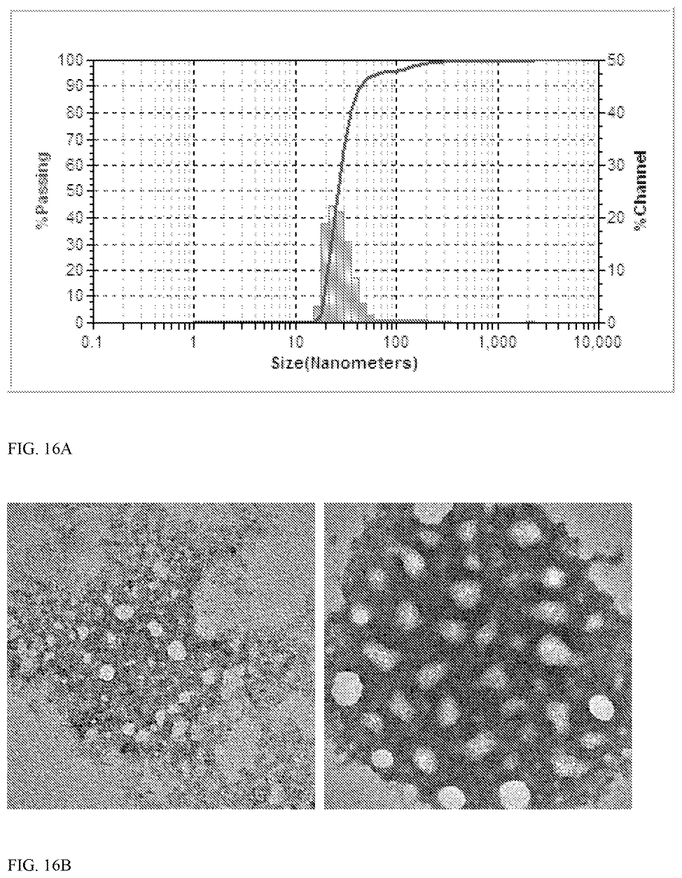

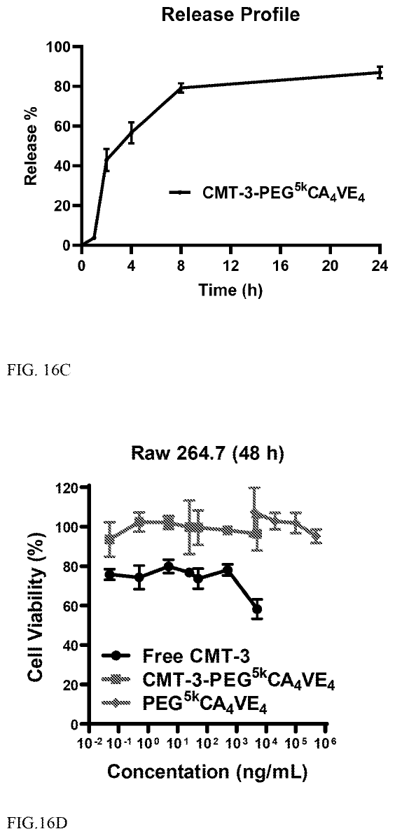

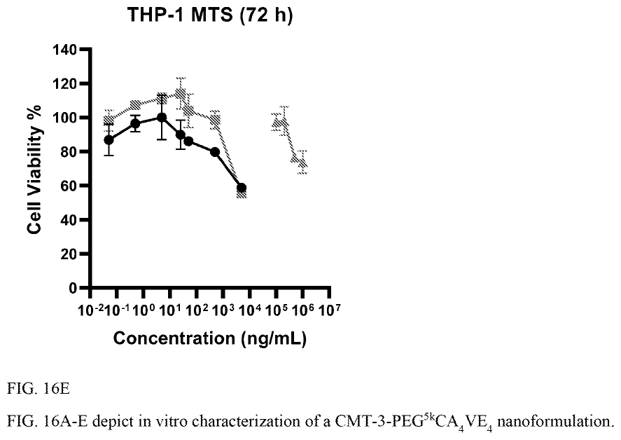

Effect of telodendrimer (PEG^{5K}CA₄VE₄) alone on ALI induced by LPS in mice: To accurately estimate the effect of nCMT-3 on lung injury from CMT-3 rather than telodendrimer (PEG^{5K}CA₄VE₄) (Nano), an experiment was designed involving the use of Nano in lung injury group. Leukocytes (neutrophil to lymphocyte ratio, NLR (%)) were counted and examined proinflammatory IL-6 and neutrophil/macrophage levels in BALF. It was found that pretreatment of Nano had no significant attenuation on increased blood NLR (%), IL-6 and neutrophil/macrophage in BALF (See FIGS. 14A and 14B) suggesting that the improvement of lung injury by using nCMT-3 was mainly due to the pharmacological effect of CMT-3 in the current study.

More specifically, FIGS. 14A and 14B depict the effect of telodendrimer (PEG^{5K}CA₄VE₄) (Nano) on neutrophil in LPS-induced lung injury in mice. Here, mice were treated with Nano-CMT-3 (1 mg/kg)/Nano (the amount of Nano is the same as in Nano-CMT-3) or vehicle 2 h before induction of lung injury by LPS (2.5 mg/kg) or sham lung injury (by saline) using non-invasive tracheal installation by aerosolizer. Mice were sacrificed 24 h after LPS or saline, then BALF was collected for Neutrophil counting. Representative Hema3-stained image for neutrophil (blue arrows) showed that no significant induction of the number of neutrophil by Nano was noted in LPS-induced lung injury.

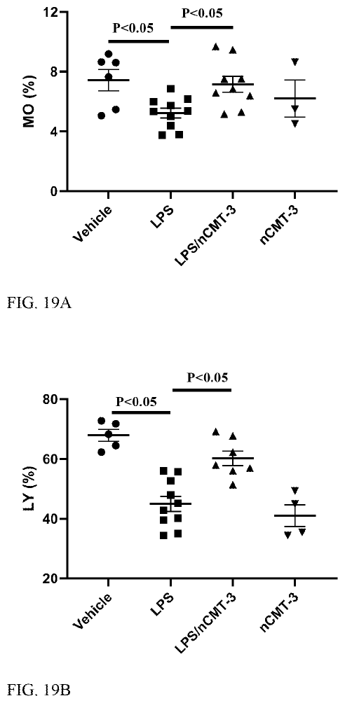

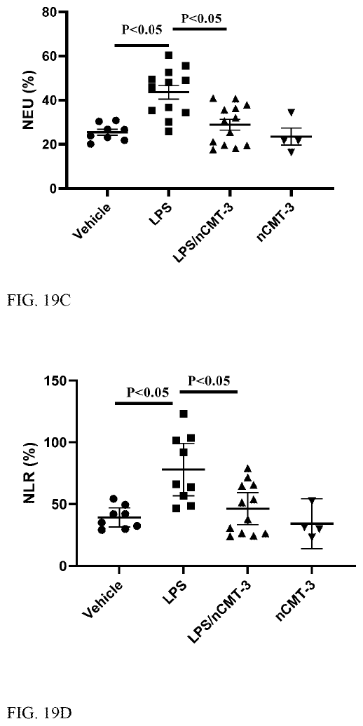

Other data that supplements the present disclosure is provided in hematological analysis shown in FIGS. 19A-19D. Here, mice were treated with Nano-CMT-3 (1 mg/kg) or vehicle 2 h before induction of lung injury by LPS (2.5 mg/kg) or sham lung injury (by saline) using non-invasive tracheal installation by aerosolizer. Mice were sacrificed 24 h after LPS or saline, then blood was collected for blood cell counting. Percentage of monocytes (MO %) (FIG. 19A), percentage of lymphocytes (LY %) (FIG. 19B), percentage of polymorphonuclear leukocytes (PMNs %) (FIG. 19C) and neutrophil-to-lymphocyte ratio (NLR %) (FIG. 19D) were calculated. Scatter dot plot represents mean values and standard error of mean (SE) (n=3-12/group).

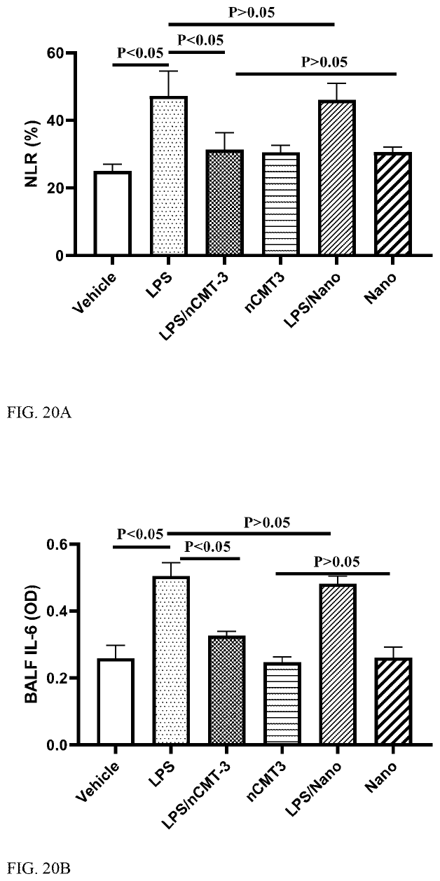

Further, FIGS. 20A and 20B depict the effect of telodendrimer (PEG^{5K}CA₄VE₄) (Nano) on NLR (%) and IL-6 in LPS-induced lung injury in mice. Here, mice were treated with Nano-CMT-3 (1 mg/kg)/Nano (the amount of Nano is the same as in nano-CMT-3) or vehicle 2 h before induction of lung injury by LPS (2.5 mg/kg) or sham lung injury (by saline) using non-invasive tracheal installation by aerosolizer. Mice were sacrificed 24 h after LPS or saline, then blood and BALF were collected for NLR (%) by counting and IL-6 by ELISA. Bar graph represents mean values and standard error of mean (SE) (n=3-7/group).

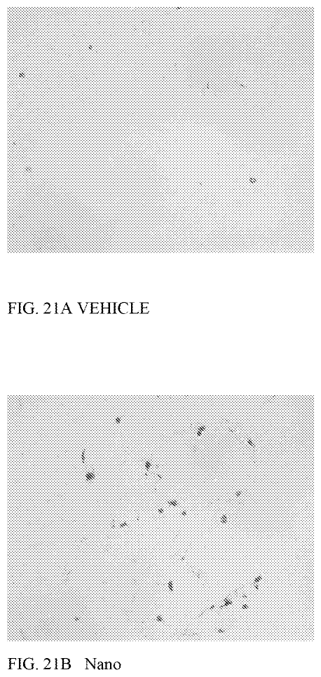

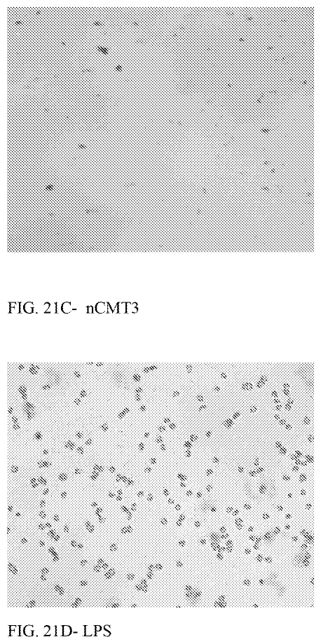

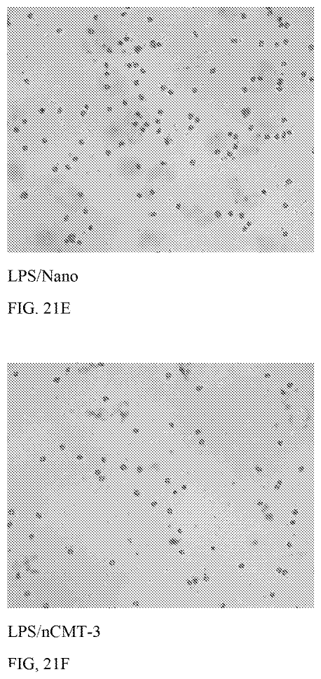

Moreover, FIGS. 21A-21F depict the effect of telodendrimer (PEG^{5K}CA₄VE₄) (Nano) on neutrophil in LPS-induced lung injury in mice. In embodiments, mice were treated with Nano-CMT-3 (1 mg/kg)/Nano (the amount of Nano is the same as in Nano-CMT-3) or vehicle 2 h before induction of lung injury by LPS (2.5 mg/kg) or sham lung injury (by saline) using non-invasive tracheal installation by aerosolizer. Mice were sacrificed 24 h after LPS or saline, then BALF was collected for Neutrophil counting. Representative Hema3-stained image for neutrophil (blue arrows) showed that no significant induction of the number of neutrophil by Nano was noted in LPS-induced lung injury.

After decades of struggle, no cure has been found for ALI and ARDS. A Nano formulation of CMT-3 is developed that can be delivered to the lung directly, thereby maximizing efficacy and enhancing resolution of inflammation while reducing collateral damage to other organs when treating lung injuries like ALI and ARDS. CMT-3 has been used to treat experimental lung injury like ALI and ARDS in animal models except for the use of cancer and oral inflammatory disease treatment. The current study, a novel Nano-formulation of CMT-3 was used through intratracheal injection (or a local administration route) to prevent lung injury in LPS-induced murine model of lung injury. The main results include: (1) nCMT-3 inhibits MMP 2 & 9 activities, decreases the levels of neutrophil elastase, modulates inflammation, blocks neutrophil infiltration and macrophage activation, and improves lung injury in LPS-induced ALI. (2) nCMT-3 down-regulates sTREM-1 expression and NLRP3 inflammasome/caspase-1 pathways in LPS-induced ALI. The findings not only provide evidence for the effectiveness of CMT-3 by local administration using Nano-formulation, but also provide new insights into the mechanism of action of CMT-3.

Several lines of evidence have led to the conclusion that CMT-3 improves lung injury and reduces mortality in direct and indirect ALI/ARDS in a variety of experimental animal models. The results have shown that various inflammatory mediators and infiltration of inflammatory cells are inhibited due to its inhibitory properties of MMP 2 & 9. Consistent with previous studies examining the effects of CMT-3 on lung injury, we show that nCMT-3 results in attenuating MMP 2 & 9 activities, levels of neutrophil elastase, inflammatory cytokines (TNF-α, IL-1β, IL-6 and IL-18), leukocytes in blood (monocyte, lymphocyte and neutrophil), the amount of protein and numbers of neutrophil/macrophage in BALF, and histological lung injury score in murine lung injury model by LPS.

As an inhibitor of MMPs, CMT-3 has pleiotropic anti-inflammatory properties. To date, the signaling pathway/molecular targets by which CMT-3 alleviate lung injury have not been well investigated, although some studies have been conducted. Since Weiss has identified an MMP-9 cleavage site in TREM-1, one is encouraged to continue to explore the molecular targets of CMT-3. TREM-1 is an immunoglobulin cell surface receptor. Blockade of TREM-1 has been shown to improve survival and modulating inflammatory response in sepsis^{33,34} because TREM-1 amplifies inflammation by regulating NF-κB, TLR-ligands, prostaglandins and Bcl-2. The effect of tetracycline and CMT-3 in inhibiting NF-κB signaling has been investigated in LPS-stimulated injury model. TREM-1 expression has been found on the surfaces of neutrophils, mature monocytes, macrophages and non-myeloid cells, such as epithelial and endothelial cells. The extracellular domain can be detected in the body fluids as soluble TREM-1 and is proposed to act as an endogenous decoy receptor and binds TREM-1 ligands, which prevents their engagement to membrane-bound TREM-1. sTREM-1 is a diagnostic and prognostic biomarker in patients with septic shock. In our study, we examined the levels of sTREM-1 in plasma and BALF, showing that nCMT-3 significantly reduced the increased sTREM-1 by LPS-induced lung injury. Although the fine-tuning mechanism has not yet been investigated in this study, our findings have revealed that the use of CMT-3 to inhibit MMP-9, leading to a reduction in sTREM-1 levels may be another novel mechanism to ameliorate lung injury. Detailed investigation will be conducted in future studies.

The activation of NLRP3/Caspase-1 signals is a major contributor in the development of ARDS. NLRP3 inflammasomes are key to host immune defense against bacterial, fungal, and viral infections. NLRP3 inflammasomes mainly exists in immune and inflammatory cells (e.g. macrophages, monocytes, dendritic cells, and splenic neutrophils) and are activated by inflammatory stimulation. Activation of the NLRP3 inflammasome by PAMPs or DAMPs (e.g. LPS) via TLRs-NF-κB signaling increases active NLRP3, pro-IL-1β, pro-IL-18, and the subsequent assembly of NLRP3, ASC (Apoptosis-associated speck like protein containing a caspase recruitment domain), and procaspase-1 into a complex which triggers the conversion of procaspase-1 to caspase-1, as well as secretion of mature IL-1β and IL-18. The success of tetracycline in treating ARDS patients by inhibiting caspase-1 activation directly (not including NLRP3 activation)⁵⁰ led us to hypothesize that CMT-3 might have a similar effect on lung injury. Tetracycline reduces high IL-1β and IL-18 levels in the patients with direct ARDS ex vivo. Additionally, CMT-3 significantly suppressed IL-1β production in alveolar leucocytes isolated from patients within 24 h of the onset of direct ARDS. Therefore, they believe that tetracycline as an immunomodulatory drug is worthy of clinical evaluation for patients with direct ARDS. On the basis of observations, direct lung injury by LPS in murine models was used to examine the levels of NLRP3, caspase-1 and proinflammatory cytokines (IL-1β and IL-18). The findings indicating that CMT-3 decreases elevated levels of NLRP3 and caspase-1 by LPS in lung tissues, as well as IL-1β in BALF and IL-18 in plasma and BALF. Our findings have revealed that both of NLRP3 and caspase-1 are reduced by NCMT-3. A study using tetracycline derivate minocycline by Lu in ischemia-induced brain damage shows similar results to ours. The activation of NLRP3 inflammasome triggers caspase-1 activation and IL-1β/IL-18 secretion, eventually resulting in an inflammatory and pyroptotic cell death. Our next step of study will focus on NLRP3/caspase-1 pathways to identify the precise target of CMT-3.

Previous studies have shown that TREM-1 activation increases LPS-induced IL-1β in human monocytes via activation of the NLRP3 inflammasome and TREM-1 aggravates inflammation in ALI by activating NLRP3 inflammasome with the involvement of NF-κB activation. Another study has demonstrated that conditioned media from NLRP3 inflammasome-activated macrophages increases TREM-1 expression by HMGB1 and IL-18 through the activation of ROS-NF-κB signals. In addition, inhibition of NLRP3 inflammasome reduces lung injury and TREM-1 expression in mice. This evidence suggests that CMT-3's effect on TREM-1 reduction is not only through direct inhibition of MPP-9 by CMT-3, but also via indirect effect on NLRP3/NF-κB activation.

The studies evidence that the Nano formulation of CMT-3 can attenuate LPS-induced lung injury by inhalation. The mechanisms for this appear to be multifactorial and potentially include reductions in TREM-1 and NLRP3 inflammasome/caspase-1 signals. Increases in MMP 2 & 9 neutrophil elastase and proinflammatory cytokines can be attenuated by free CMT-3 in lung injury. Consistent with these mechanisms, increases of the above markers were observed in LPS-induced lung injury were ameliorated by nCMT-3. The highlights of this study are that nCMT-3 attenuated the increase in TREM-1 and deactivated NLRP3/caspase-1 signals. Collectively these data provide evidence that nCMT-3 can attenuate lung injury via inhibiting MMP 2 & 9 neutrophil elastase as well as down-regulating TREM1 and NLRP3/caspase-1 signals during lung injury.

Despite the excellent findings herein, there are several limitations to the results. First, the ALI model has several limitations in common. Most models are based on one or at most two methods to induce injury, but human ARDS is associated with a complex interaction between major risk factors and comorbidities. The conceptual model of ARDS includes lung inflammation, acute severe hypoxemia, edema, hyaline membranes, and alveolar hemorrhage. One of the major pitfalls to translation research is that none of the ALI animal models adequately reproduces the full characteristics of human ARDS although the model of ALI induced directly by intratracheal instillation of LPS reproduces more effectively the characteristics of the acute phase of human ARDS, including microvascular lung injury, including accumulation of leukocyte (deformability/entrapment in pulmonary capillaries and large increases in neutrophils in the air spaces) in lung tissue, pulmonary edema and inflammation, as well as increased albumin and proinflammatory cytokines in BALF after intratracheal instillation of LPS at an early stage (<24 h). Second, the effects of different time windows on nCMT-3 was not fully characterized in the studies and pre-injury dosing was used. However, in the preliminary experiment, it was found that administration of nCMT-3 2 h after lung injury still reduced the level of proinflammatory cytokines (IL-6) in BALF (see Data in FIGS.). In addition, a lethal dose of LPS to observe the effect of nCMT-3 on mortality was not used, as the focus of the study is to explore, inter alia, the mechanism and to study the new CMT-3 formulation using a local delivery route through the lungs.

### Example III

Materials and Methods: Male and female pigs (35-40 kg) will be anesthetized and intubated. nCMT-3 (1 mg/kg) was administered using a clinically available aerosolization system (AirLife™ Nebulizer, Vyaire Medical, Inc., IL, USA). Animals were monitored for 24 hours with blood samples obtained at 0.5, 1, 2, 4, 8, 12, 18, and 24 hours to determine the PK. At 24 hours, animals were euthanized and necropsy performed. Based on the preliminary data of nCMT-3 bioavailability, the half-life of nCMT-3 is approximately 6 hours.

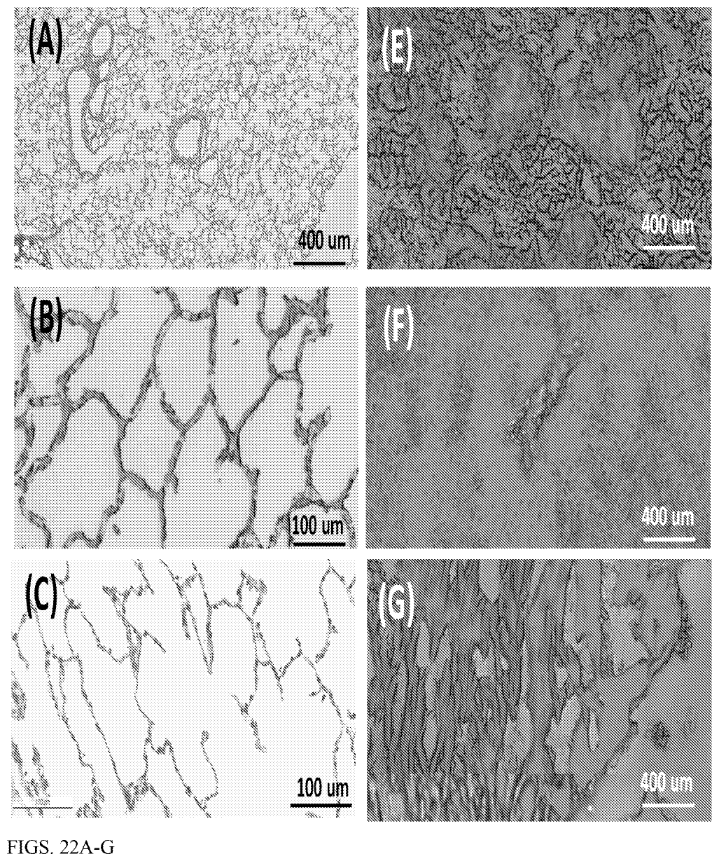

Experiments were performed to demonstrate that embodiments of the present disclosure such as nCMT-3 can be aerosolized and appropriately deposits in the target alveoli. Thus, a nanoparticle of the present disclosure was labeled by conjugation with a fluorescent dye (Rhodamin B), then encapsulated CMT-3 for aerosolization and deposition studies in mechanically ventilated healthy pigs was performed. A 4 mL dose of nCMT-3 (25 μg/kg) solution was nebulized almost completely (>90%) after 10 minutes. Organs were harvested 15 minutes and three hours after nebulization for tissue fluorescence imaging and histology studies. The orange fluorescent labeled nCMT-3 homogenously distributed through the lung and deep into alveoli, indicating efficient drug delivery (See FIGS. 22A-22B). The lung tissue was also evaluated using H&E staining and microscopy and revealed no tissue damage or inflammatory reaction (See FIG. 22C). Significant fluorescent signal was also observed in the kidney, liver, and heart (See FIGS. 22E-22G wherein higher intensity is due to denser tissue in these organs). These findings indicate protective effects of aerosolized nCMT-3 for other vital organs in ARDS and sepsis.

More specifically, FIGS. 22A-22G depict aerosolized nCMT-3 delivery to pig lungs. Organs were harvested 15 minutes after aerosolization of nCMT-3 labeled with Rhodamin B fluorescent dye and demonstrated homogeneous and deep deposition within the large airways (FIG. 22A) and target alveoli (FIG. 22B). H&E staining revealed normal lung structure without noticeable damage induced by nCMT-3 (FIG. 22C). Fluorescent imaging also revealed nCMT-3 accumulates in the kidney (FIG. 22E), liver (FIG. 22F), and heart (FIG. 22G), indicating protective effects of nCMT-3 on remote organs.

Many modifications and other embodiments of the disclosure will come to mind to one skilled in the art to which this disclosure pertains having the benefit of the teachings presented in the foregoing descriptions and the associated drawings. Therefore, it is to be understood that the disclosure is not to be limited to the specific embodiments disclosed herein and that modifications and other embodiments are intended to be included within the scope of the appended claims. Although specific terms are employed herein, they are used in a generic and descriptive sense only and not for purposes of limitation.

1-18. (canceled)

19. A nanocarrier composition, comprising:
one or more telodendrimers suitable for binding to one or more tetracyclines; and one or more tetracyclines have substantially no antibacterial activity.

20. The nanocarrier composition of claim 19, wherein the tetracycline is 6-demethyl-6-deoxy-4-de(dimethylamino)tetracycline (CMT-3), or a derivative thereof.

21. The nanocarrier composition of claim 19, wherein the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof.

22. The nanocarrier composition of claim 19, wherein the one or more telodendrimers comprise a formula:

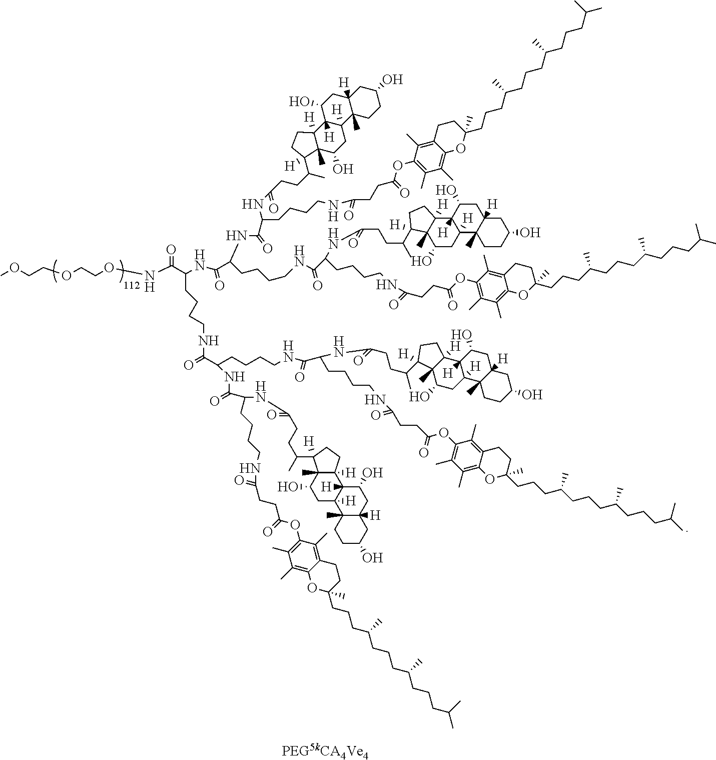

23. The nanocarrier composition of claim 19, wherein the one or more telodendrimers comprise a formula:

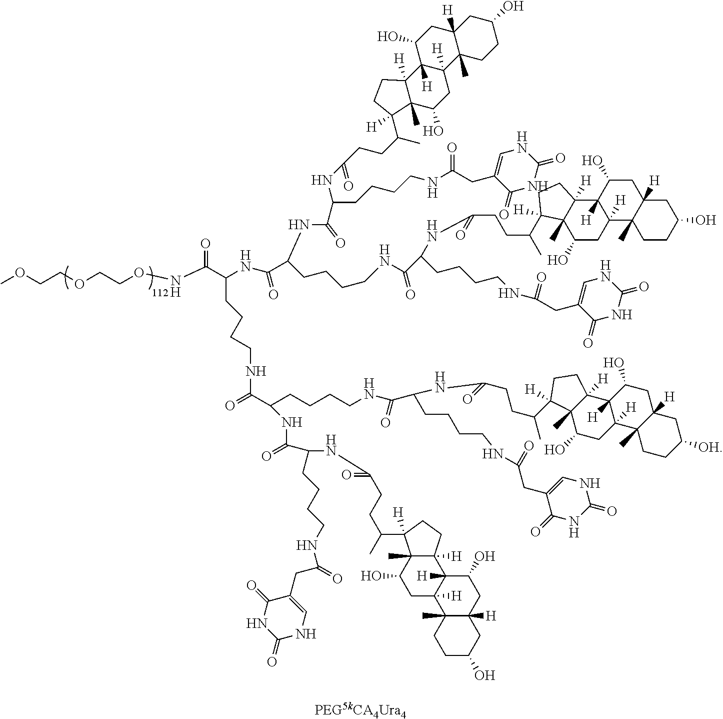

24. The nanocarrier composition of claim 19, wherein the one or more telodendrimers comprise a formula:

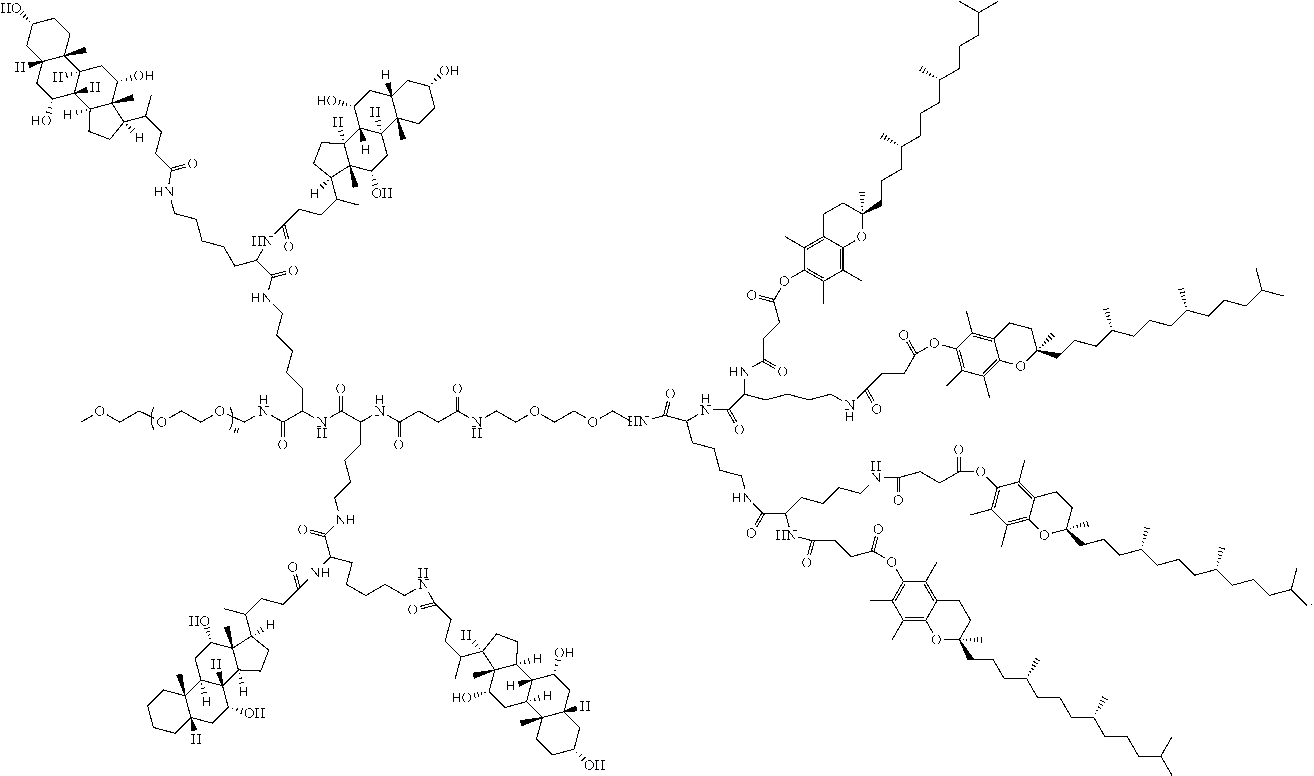

25. The nanocarrier composition of claim 19, wherein the one or more telodendrimers comprise one or more of a PEG^{nk} moiety (such as wherein n=2-40, k is kDa), a two layered poly-lysine moiety, a three-layered poly-lysine moiety, a CA (cholic acid) moiety, a vitamin E (VE) moiety, a URA moiety, a uracil-5-ylacetic acid moiety, a (2,4-dioxide-1,2,3,4-tetrahydro-5-pyrimidinyl) acetic acid moiety, or combinations thereof.

26. The nanocarrier composition of claim 19, wherein the composition is characterized by self-assembly drug loading.

27. The nanocarrier composition of claim 19, wherein CMT-3 is physically bound to the one or more telodendrimers.

28. A method for preventing, alleviating, or treating acute lung injury in a mammal, the method comprising:
administering a nanocarrier composition to a mammal in need thereof in a therapeutically effective amount, wherein the nanocarrier composition comprises one or more telodendrimers suitable for binding to one or more tetracyclines, wherein the one or more tetracyclines have substantially no antibacterial activity.

29. The method of claim 28, wherein the tetracycline is 6-demethyl-6-deoxy-4-de(dimethylamino)tetracycline (CMT-3), or a derivative thereof.

30. The method of claim 28, wherein the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof.

31. The method of claim 28, wherein the one or more telodendrimers comprise a formula:

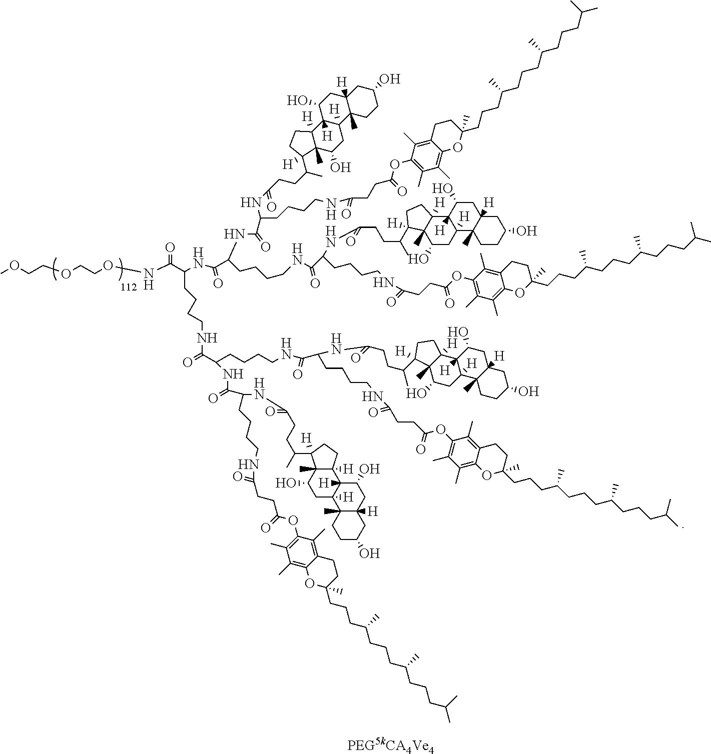

32. The method of claim 28, wherein the one or more telodendrimers comprise a formula:

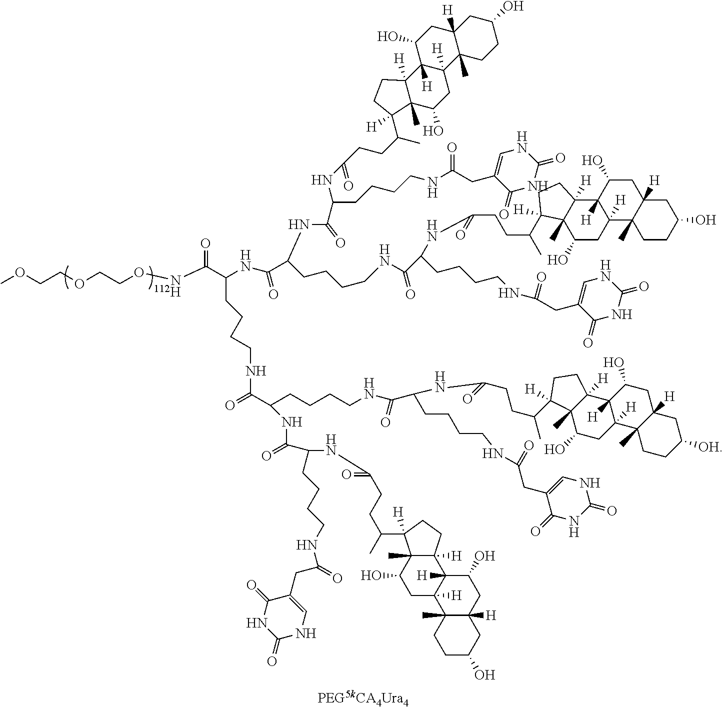

33. The method of claim 28, wherein the one or more telodendrimers comprise a formula:

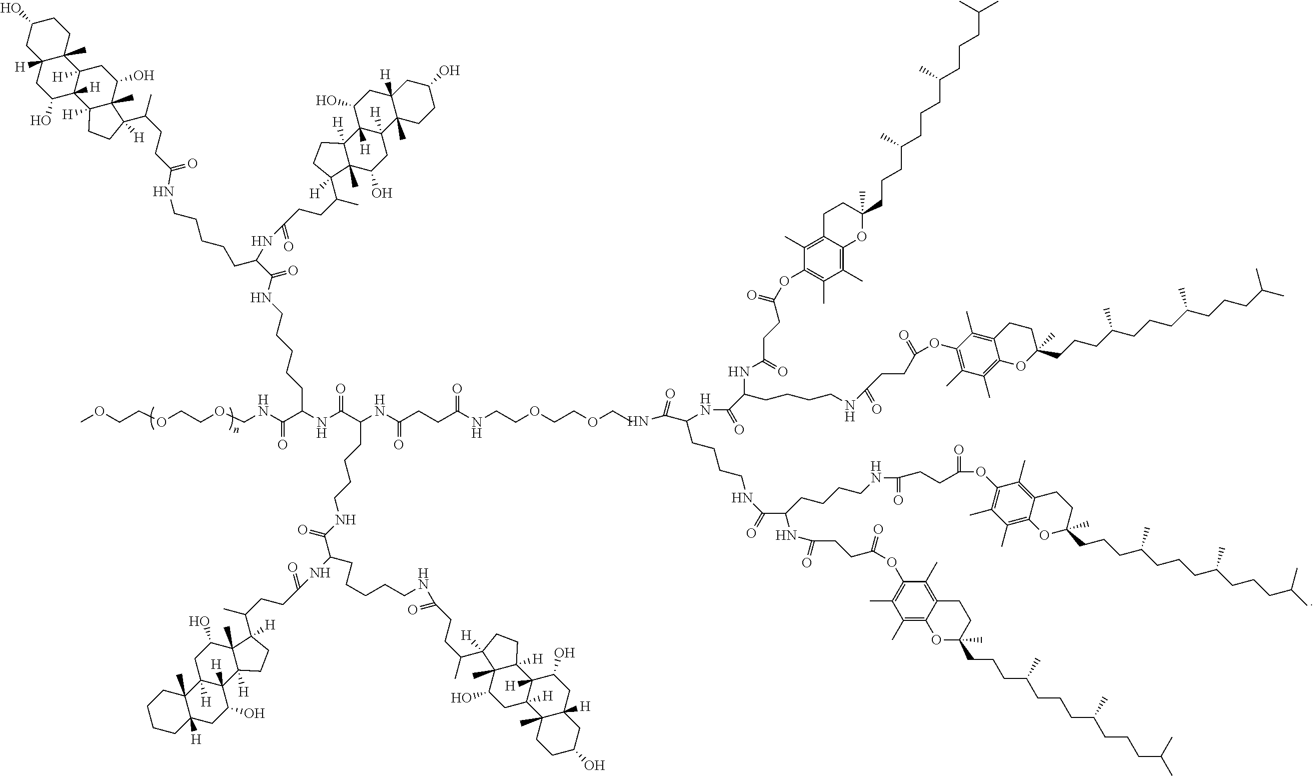

34. The method of claim 28, wherein the one or more telodendrimers comprise one or more of a PEG^{nk} moiety (such as wherein n=2-40 kDa), a two layered poly-lysine moiety, a three-layered poly-lysine moiety, a CA (cholic acid) moiety, a vitamin E (VE) moiety, a URA moiety, a uracil-5-ylacetic acid moiety, a (2,4-dioxide-1,2,3,4-tetrahydro-5-pyrimidinyl) acetic acid moiety, or combinations thereof.

35. The method of claim 28, wherein the composition is characterized by self-assembly drug loading.

36. The nanocarrier composition of claim 19, wherein the nanocarrier composition comprises: one or more telodendrimers suitable for binding to a tetracycline, wherein the tetracycline is 6-demethyl-6-deoxy-4-de(dimethylamino)tetracycline (CMT-3), or a derivative thereof, and wherein the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof.

37. A method of treating inflammation or inflammatory disease comprising:
administering a nanocarrier composition of claim 19 to a mammal in need thereof in a therapeutically effective amount.

38. The method of claim 37, wherein the nanocarrier composition targets, dental, lung, liver, kidney tissues, or combinations thereof.

39. The method of claim 37, wherein the tetracycline is 6-demethyl-6-deoxy-4-de(dimethylamino)tetracycline (CMT-3), or a derivative thereof.

40. The method of claim 37, wherein the one or more telodendrimers suitable for binding to one or more tetracyclines are selected from the group consisting of PEG^{5k}CA₄Ve₄, PEG^{5k}CA₄Ura₄, PEG^{nk}CA₄-L-VE₄, or combinations thereof.

41. A pharmaceutically acceptable composition for pulmonary inhalation, comprising: one or more nanocarriers comprising one or more telodendrimers suitable for binding to one or more tetracyclines, wherein the one or more tetracyclines have substantially no antibacterial activity.

42. The pharmaceutically acceptable composition of claim 41, wherein the composition is suitable for aerosolized delivery to a subject in need thereof.

43. The pharmaceutically acceptable composition of claim 41, wherein the composition is a dry powder.

44. A pharmaceutically acceptable composition for injectable administration, comprising: one or more nanocarriers of claim 19.

45. The pharmaceutically acceptable composition of claim 44, wherein the composition is a liquid, and comprises a therapeutically effective amount of tetracycline.

46. The pharmaceutically acceptable composition of claim 45, wherein the tetracycline is characterized as an anti-inflammatory.
